(19) United States
(12) Patent Application Publication
(10) Pub. No.: US 2003/0008273 A1
(43) Pub. Date: Jan. 9, 2003

(54) Methods for increasing the survival of neuronal cells

(76) Inventors: Thomas Perlmann, Sollentuna (SE); Asa Wallen, Stockholm (SE); Ernest Arenas, Stockholm (SE); Joe Wagner, West Chester, PA (US)

Correspondence Address: FULBRIGHT & JAWORSKI, LLP, 666 FIFTH AVE, NEW YORK, NY, 10103-3198, US

(21) Appl. No.: 10/166,158

(22) Filed: Jun. 11, 2002

Related U.S. Application Data
(60) Provisional application No. 60/296,729, filed on Jun. 11, 2001.
(60) Provisional application No. 60/313,463, filed on Aug. 21, 2001.
(60) Provisional application No. 60/372,086, filed on Apr. 15, 2002.

(51) Int. Cl. A61K 31/203; C12Q 1/00

(52) U.S. Cl. 435/4; 514/559

(57) ABSTRACT

The invention relates to methods of increasing the survival of dopamine secreting cells, comprising the steps of administering a retinoid X receptor (RXR) ligand to dopamine secreting cells, wherein the binding of the RXR ligand increases the survival of the dopamine secreting cells. Therapeutic approaches to neurodegenerative diseases are also disclosed.

### FIELD OF THE INVENTION

This invention also relates to the treatment of neurodegenerative diseases by increasing the survival of dopamine secreting cells. Disclosed herein is the surprising discovery that RXR ligands can increase the survival of dopamine secreting cells by activating a cell survival pathway mediated through the RXR-ligand dependent activation of Nurr1. This discovery provides the basis for therapeutic methods of increasing the survival of dopamine secreting cells comprising administering RXR ligand to a dopamine secreting cell. Based on the same principle, this discovery provides the basis for methods of identifying genes that are transcriptionally activated by RXR following binding of an RXR ligand and that mediate the RXR-dependent increase in dopamine secreting cell survival. The inventors of the present invention have also surprisingly discovered that Nurr1 regulates the expression of the receptor-tyrosine kinase, Ret, in both dopamine-secreting cells and cells of the dorsal-motor nucleus of the vagus nerve.

### BACKGROUND AND PRIOR ART

1. Parkinson's Disease

Midbrain dopamine-secreting cells are critical for control of voluntary motor functions, motivation and other neural functions. It is believed that dysfunctional dopamine neurotransmission causes disorders such as schizophrenia and drug addiction. Importantly, the deregulation and loss of dopamine-secreting cells is associated with Parkinson's disease, a neurodegenerative disease that affects more than 2% of the population in North America. The primary pharmaceutical treatments of Parkinson's disease focus on increasing dopamine levels by providing exogenous dopamine precursors and inhibiting peripheral dopamine-degrading enzymes (Dunnett et al. (1999) Nature 399, A332-A39). Although dopamine-replacement therapy can successfully ameliorate the symptoms of Parkinson's disease, no current treatment can either block or slow neurodegeneration and the loss of dopamine-secreting cells in these patients. In addition, dopamine-replacement therapy is associated with numerous side effects and the development of tolerance.

Neuronal grafts of embryonic dopamine-secreting cells have been successfully used in treating experimentally-induced Parkinsonism in rodents, primates and human Parkinsonian patients. Implants of embryonic mesencephalic tissue containing dopamine cells, into the caudate and putamen of human patients, was shown to offer long-term clinical benefit to some patients with advanced Parkinson's Disease. (Freed et a. (1992) N. Engl. J. Med. 327: 1549-1555). Long-term functional improvements have also been demonstrated in patients with MPTP-induced Parkinsonism (MPTP is 1-methyl-4-phenyl-1,2,3,6-tetrahydropyridine) that received bilateral implantation of fetal mesencephalic tissue. (Perlow et al. (1978) Science 204: 643-647; Spencer et al. (1992) N. Engl. J. Med. 327: 1541-1548; Widner et al. (1992) N. Engl. J. Med. 327: 1556-1563). Graft survival, however, is poor and only limited quantities of embryonic dopaminergic tissue are available. On average, 4-10 fresh, human embryos are required to obtain sufficient numbers of dopaminergic neurons for a single human transplant. (Widner et al. (1992) N. Engl. J. Med. 327: 1556-1563).

While the studies noted above are encouraging, the use of large quantities of aborted fetal tissue for the treatment of disease raises ethical considerations and political obstacles. There are other considerations as well. Tissue from the fetal central-nervous system is composed of more than one cell type, and thus, is not a well-defined source of tissue. In addition, there are serious doubts regarding the supply of fetal tissue. Potential viral and bacterial contamination of fetal tissue, during and prior to preparation, raises the need for expensive diagnostic testing. A preferred treatment would involve prevention or reduction in cellular degeneration.

2. Dopamine-Secreting-Cell Survival

Several methods for increasing dopamine-secreting-cell survival that use polypeptide neurotrophic factors have been developed. In animal models of Parkinson's disease, numerous polypeptide factors, including glial-cell-line-derived neurotrophic factor (GDNF), neurturin, basic- fibroblast growth factor (bFGF), brain-derived neurotrophic factor (BDNF), neurotrophins 3 and 4/5, cilliary neurotrophic factor and transforming growth factor, increase the survival of nigral dopamine-secreting cells, in vivo. (Bjorklund et al. (2000) Nature 405: 892-893; Magavi et al. (2000) Nature 405: 951-955; Unsicker et al. (1996) Ciba Found. Symp. 196: 70-80; Gash et al (1998) Ann. Neurol. (suppl) 44: S121-S125; Bjorklund et al. (1997) Neurobiol. Dis. 4: 186-200; Palfi et al. (1998) Soc. Neurosci. Abstr. 24: 41; Tomac et al. (1995) Nature 373: 335-339; Beck et al. (1995) Nature 373: 339-341; Yan et al. (1995) Nature 373: 341-344). These polypeptide factors, however, do not cross the blood-brain barrier and thus, do not reach the target cells. Stereotactic injection of viral vectors encoding GDNF into the substantia nigra may overcome this problem. (Bjorklund et al.(2000) Nature 405: 892-893).

3. Nuclear Receptors-RXR

Nuclear receptors consist of a family of highly related proteins that are regulated by small molecule ligands such as steroid hormones, thyroid hormone, retinoids and vitamin D (Mangelsdorf et al. (1995) Cell 83: 835-850). These intracellular receptors function as ligand activated transcription factors that bind small lipophilic ligands, which cross the blood-brain barrier more effectively than polypeptide hormones and factors. The nuclear receptors are characterized by a variable N-terminal region, a conserved central DNA-binding domain, a variable-hinge region, a conserved C-terminal ligand-binding domain, and a variable C-terminal domain. (Mangelsdorfet al. (1995) Cell 83: 835-850). The DNA-binding domain comprises two highly conserved zinc fingers that enable the receptor to bind specific DNA sequences referred to as hormone-response elements. Ligand specificity and selectivity is conferred upon the receptor by the ligand-binding domain. Although the DNA-binding domain of nuclear receptors is generally sufficient for DNA binding, ligand binding is necessary for transcriptional activation of a target gene. Ligand-mediated activation of the nuclear receptor is necessary but insufficient for transcriptional upregulation, which involves an additional series of complex events, including the recruitment of several coactivating factors that bind the promoter of the target gene.

Nuclear receptors are often classified according to their ligand binding, DNA binding and dimerization properties. The steroid-hormone receptors, for example, form homodimers that bind DNA half-sites that are organized as inverted repeats. In fact, dimers represent the functional form of most nuclear receptors. The retinoid receptors, comprised of the retinoic acid receptors (RARs) and the retinoid X receptors (RXRs), function as heterodimers that bind direct DNA repeats. (Kastner et al. (1995) Cell 83: 859-869; Mangelsdorf et al. (1995) Cell 83: 835-850). RXRs have been shown to form heterodimers with a large number of nuclear receptors, including RARs and several orphan receptors, a subset of the nuclear-receptor family for which no known ligands have been identified. (Gigere et al. (1999) Endocrine Rev. 20: 689-725; et al. (1995) Cell 83: 859-869). Exclusive of the steroid-hormone receptors, all other known ligand-dependent receptors form heterodimers with RXR. Although RXR homodimers have been identified (Lehmann et al. (1992) Science 258:1944-1946; incorporated herein by reference), it has been postulated that these structures are artifacts arising from in vitro overexpression of RXR. The physiological significance of these homodimers remains unclear.

The fact that RXR is a heterodimerization partner of many nuclear receptors that mediate numerous physiological responses suggests that RXR is a potentially versatile therapeutic target. Furthermore, activation of an RXR-nuclear receptor heterodimer to the transcriptionally active state can be induced by the binding of either an RXR ligand or a ligand of the RXR-heterodimerization partner. RXR-mediated responses have been implicated in embryonic development, cellular function and disease. For example, RXRs are essential for embryonic development and normal central-nervous system function in adult mice. In addition, the activation of an RXR-dependent-response pathway appears therapeutically beneficial in treating diabetes and breast cancer. (Mukherjee et al. (1997) Nature 386: 407-410; Gottardis et al. (1996) Cancer Research 56: 5566-5570).

Any potential therapy that is based on the activation of an RXR-dependent-response pathway requires an RXR ligand that: 1) binds RXR; 2) induces the RXR heterodimer to assume a transcriptionally active conformation; and 3) ultimately upregulates the transcription of the target gene. Several naturally-occurring compounds (i.e., 9-cis retinoic acid, docosahexacnoic acid, all-trans retinoic acid) and non-naturally-occurring compounds have been identified as RXR and RAR ligands. Two vitamin A derivatives-all-trans retinoic acid and 9-cis retinoic acid-are RAR ligands and activate RAR. In contrast, RXR is activated by 9-cis retinoic acid and not all-trans retinoic acid. In addition to 9-cis retinoic acid, several non-naturally-occurring RXR ligands and RXR-ligand analogs have been developed that selectively bind and activate RXR heterodimers, but do not substantially bind RAR.

4. Nurr1 and Dopamine-Secreting-Cell Survival

Nurr1 (nur-related factor 1; NR4A2) is an orphan-nuclear receptor that is widely expressed in both the developing and adult central-nervous system (Law et al. (1992) Mol. Endocrinol. 6: 2129-2135; Zetterström (1996) Mol. Endocrinol. 10: 1656-1666; Zetterström et al. (1996) Mol. Brain Res. 41: 111-120). Nurr1 expression is detected at embryonic day E10.5 in the ventral midbrain (VMB) of mice, which is the region where mesencephalic dopamine-secreting cells develop. (Zetterström et al. (1997) Science 276: 248-250). Previous studies in Nurr1-knockout mice (Nurr1^{-/-}) revealed that Nurr1 is essential for the development of dopamine-secreting cells. (Zetterström et al. (1997) Science 276: 248-250; Saucedo-Cardenas et al. (1998) Proc. Natl. Acad. Sci. USA 95: 4013-4018; Castillo et al. (1998) Mol. Cell. Neurosci. 11: 36-46). Analyses demonstrated that Nurr1, which is first detected immediately peripheral to the ventricular zone of proliferating progenitor cells, is required for dopamine-secreting-cell migration and axonal-target-area innervation. (Wallën et al. (1999) Exp. Cell Res. 253: 737-746). At birth, increased cell death is observed in the ventral midbrain of Nurr1 -deficient mice and dopaminergic markers fail to be detected. (Saucedo-Cardenas et al. (1998) Proc. Natl. Acad. Sci. USA 95: 4013-4018; Wallën et al. (1999) Exp. Cell Res. 253: 737-746; Zetterström et al. (1997) Science 276: 248-250). Nurr1 is also expressed in adult dopamine-secreting cells suggesting that Nurr1 continues to influence the function of these cells during postnatal development and adulthood. (Zetterström et al. (1996) Mol. Endocrinol. 10: 1656-1666; Zetterström (1996) Mol. Brain Res. 41: 111-120). Indeed, Nurr1 has been shown to regulate genes of importance for dopaminergic neurotransmission, including the dopaminergic transporter and tyrosine hydroxylase (TH). (Sacchetti et al. (2001) J. Neurochem. 76: 1565-1572; Sakurada et al. (1999) Development 126: 4017-4026; Schimmel et al. (1999) Brain Res. Mol. Brain Res. 74: 1-14; Zetterström (1997) Science 276: 248-250).

Recently, proteins important for dopaminergic neurotransmission, including tyrosine hydroxylase (TH) and dopamine transporter, were shown to be regulated by Nurr1 in cells cultured in vitro (Sacchetti et al. (2001) J. Neurochem. 76: 1565-1572; Sakurada (1999) Development 126: 4017-4026; Schimmel et al (1999) Brain Res. Mol. Brain Res. 74: 1-14). Tyrosine hydroxylase, for example is not expressed in Nurr1^{-/-}-mutant VMB. The severe developmental phenotype characterized by loss of dopaminergic markers, defective dopamine-secreting-cell migration, target-area innervation and early-postnatal death, however, emphasizes the importance of identifying additional deregulated effector genes. (Castillo et al. (1998) Mol. Cell. Neurosci. 11: 36-46; Le et al. (1999) Exp. Neurol. 159: 451-458; Saucedo-Cardenas et al. (1998) Proc. Natl. Acad. Sci. USA 95: 4013-4018; Wallén et al. (1999) Exp. Cell Res. 253: 737-746; Zetterström (1997) Science 276: 248-250).

Ret is a protein tyrosine kinase and a critical signal transducing subunit of receptors for glial-cell-line-derived neurotrophic factor (GDNF) and related neurotrophic factors (Baloh et al. (2000) Curr. Opinion Neurobiol. 10: 103-110). Deletion of the Ret gene in mice results in early postnatal death due to developmental defects in, for example, the kidneys and peripheral nervous system. (Durbec et al. (1996) Nature 381: 789-793; Schuchardt et al (1994) Nature 367: 380-383). Ret is also expressed in dopamine-secreting cells, and has been implicated in cell-survival pathways. GDNF, a Ret ligand, and related factors, promote neuronal-cell survival in vitro and in vivo. (Beck et al. (1995) Nature 373: 339-341; Hoffer et al. (1994) Neurosci. Lett. 182: 107-111; Lin et al. (1993) Science 260: 1130-1132; Saueret al (1995) Proc. Natl. Acad. Sci. USA 92: 8935-8939; Strömberg et al. (1993) Exp. Neurol. 124: 401-412; Tomac et al. (1995) Nature 373: 335-339; Trupp et al. (1996) Nature 381: 785-789). Although dopamine-secreting cells are generated in Ret-deficient mice, signaling by Ret appears essential for correct maturation and function in mature dopamine-secreting cells. (Marcos et al. (1996) Int. J. Dev. Biol. Suppl. 1: 137s-I38S; Golden et al. (1999) Exp. Neurol.158: 504-528; Granholm et al. (2000) J. Neurosci. 20: 3182-3190; Messer et al. (2000) Neuron 26(1): 247-257; Lin et al. (1993) Science 260: 1130-1132; Strömberg et al. (1993) Exp. Neurol. 124: 401-412).

Several studies have demonstrated that Ret influences both postnatal survival and innervation of dopamine-secreting cells. (Batchelor et al. (2000) Eur. J. Neurosc.i 12: 3462-3468; Tomac et al. (1995) Nature 373: 335-339; Granholm et al. (2000) J. Neurosci. 20: 3182-3190). It is well established that signaling via Ret promotes a robust survival pathway in many cell types including VMB Dopaminergic cells (Beck et al. (1995) Nature 373: 339-341; Hoffer et al. (1994) Neurosci. Lett. 182: 107-111; Lin et al. (1993) Science 260: 1130-1132; Oppenheim et al. (1995) Nature 373: 344-346; Sauer et al. (1995) Proc. Natl. Acad. Sci. USA 92: 8935-8939; Tomac et al. (1995) Nature 373: 335-339; Williams et al. (1996) J. Pharmacol. Exp. Ther. 277: 1140-1151; Yan et al. (1995) Nature 373: 341-344). In models of Parkinson's disease in both rodents and primates, Ret ligands such as GDNF efficiently influence dopamine-secreting-cell survival and may likely become important in future therapies for Parkinson's disease (Björklund et al. (2000) Brain Res. 886: 82-98; Kordower et al. (2000) Science 290: 767-773; Olson (2000) Science 290: 723-724.).

Disclosed herein is the surprising discovery that RXR ligands can increase the survival of dopamine secreting cells by activating a cell survival pathway mediated through the RXR-ligand dependent activation of Nurr1. This discovery provides the basis for therapeutic methods of increasing the survival of dopamine secreting cells comprising administering RXR ligand to a dopamine secreting cell. Based on the same principle, this discovery provides the basis for methods of identifying genes that are transcriptionally activated by RXR following binding of an RXR ligand and that mediate the RXR-dependent increase in dopamine secreting cell survival. The inventors of the present invention have also surprisingly discovered that Nurr1 regulates the expression of the receptor-tyrosine kinase, Ret, in both dopamine-secreting cells and cells of the dorsal-motor nucleus of the vagus nerve. This Nurr1-dependent regulation of Ret-gene expression implicates Nurr1 and RXR in signaling pathways that mediate dopamine-secreting-cell survival.

### BRIEF DESCRIPTION OF THE DRAWINGS

FIG. 1 demonstrates the effects of midbrain explants on transfected-human-choriocarcinoma cells (JEG3). (A) Ventral and dorsal-midbrain explants from three different embryonic stages on JEG3 cells transfected with Nurr1, CMX-RXR and the reporter MH100-tk-luc. (B) E13.5 midbrain explants on JEG3 cells cotransfected with either wild-type Nurr1 or Nurr1 dim⁻, which is able to heterodimerize with RXR, the reporter MH100-tk-luc, and/or CMX-RXR. (C) Titration curve of 9-cis retinoic acid on JEG3 cells transfected with RAR or Nurr1 and CMX-RXR. MH100-tk-luc used as reporter. The bar-graph demonstrates the effect of overnight conditioned media from E13.5 embryo midbrain and cortex on JEG3 cells transfected with gRAR or Nurr1 and CMX-RXR. MH100-tk-luc is the reporter.

FIG. 2 demonstrates the effect of the non-naturally-occurring-RXR ligand, SR11237, on the survival of dopamine-secreting cells. (A) Primary midbrain cultures were either untreated (N2), or treated with the non-naturally-occurring-RXR agonist SR11237, the RAR agonist TTNPB, or all-trans retinoic acid (atRA). (B) Primary midbrain cultures were either untreated (N2), or treated with 0.1 μM SR11237 or 30 ng GDNF. (C) Primary midbrain cultures were either untreated (N2), or treated with 0.1 μM SR11237 and pulse labeled with bromodeoxyuridine (BrdU).

FIG. 3 depicts the structure of the non-naturally-occurring-RXR ligands or RXR-ligand analogs SR11203, SR 11217, SR11234, SR11235, SR11236 and SR11237. SR11203, wherein R and R′ are SCH₂CH₂CH₂S; S11217, wherein R and R′ are (CH3)₂C; SR11234, wherein R and R′ are SCH₂CH₂S; SR11235, wherein R and R′ are OCH₂CH₂S; SR11236, wherein R and R′ are OCH₂CH₂CH₂O; and SR11237, wherein R and R′ are OCH₂CH₂O.

FIG. 4 depicts the generic structure of the non-naturally-occurring-RXR ligands or RXR-ligand analogs. (A) Generic structure of the non-naturally-occurring-RXR ligands or RXR-ligand analogs described by Boehm et al. and Mukherjee et al. (Boehm et al. (1994) J. Med. Chem. 37: 2930-2941; Boehm et al. (1995) J. Med. Chem. 38: 3146-3155; Mukherjee et al. (1997) Nature 386: 407-410; all incorporated herein by reference). (B) Structure of LG1069 (Boehm et al. (1994) J. Med. Chem. 37: 2930-2941; Mukherjee et al. (1997) Nature 386: 407-410; both incorporated herein by reference) and LG100268 (Boehm et al. (1995) J. Med. Chem. 38: 3146-3155; Mukherjee et al. (1997) Nature 386: 407-410; both incorporated herein by reference).

FIG. 5 depicts coronal sections showing Nurr1 protein immunoreactivity in the ventral midbrain at E12.5. Using an antibody raised against the carboxy-terminal region of Nurr1, specific nuclear immunoreactivity (red) can be detected in the medial VMB outside the ventricular zone (A). A Raldh I specific antibody shows cytoplasmic immunoreactivity (green) in both the ventricular and periventricular zones in the same region (B). Double labeling for these two markers (Nurr1 in red and Raldh I in green) shows complete colocalization in the cells of the mantle layer (C and D). Costaining for Nurr1 (red) with TH (green) shows Nurr1 -nuclear expression of differentiating-dopamine-secreting cells with cytoplasmic TH immunoreactivity (E and F). Scale bars: A (also applies for B) 50 μm, C 20 μm, D 5 μm, E 50 μm, F 10 μm. 3V=third ventricle, VZ=ventricular zone, MZ=mantle zone.

FIG. 6 depicts an in situ hybridization analysis of Ret and GFRα1 mRNA in the ventral midbrain at E11.5. Adjacent coronal sections of Nurr1^{+/+} (left panel) and Nurr1^{-/-} (right panel) embryos showing expression patterns of Nurr1 (A and B; detection with the probe that recognizes also the disrupted Nurr1 transcript), TH (C and D), Ret (E and F) and GFRα1 (G and H). As previously described, TH is not detected in the Nurr1 mutant midbrain (D). In the Nurr1^{+/+}, Ret is detected in medial-ventral-midbrain dopamine-secreting cells as well as in the laterally located motor neurons (E). The Nurr1^{-/-} VMB fails to display Ret expression in the medial cells, while it can be detected in the laterally located motor neurons (F). In contrast, at this stage GFRα1 appears in the VMB cells in both the Nurr1^{+/+} (G) and Nurr1^{-/-} (H) embryos. Scale bar: 200 μm. 3V=third ventricle, VZ=ventricular zone, MZ=mantle zone, DA=DA cells, MN=motor neurons.

FIG. 7 depicts in situ hybridization analyses of the brainstem in newborn mouse. Serial coronal sections of Nurr1^{+/+} (left panel) and Nurr1^{-/-} (right panel) pups showing the mRNA expression of Nurr1 (A and B), ChAT (C and D), Phox2a (E and F), and Ret (G and H). In the newborn pup, Nurr1 can easily be detected in the DMN of the vagus nerve, whereas no labeling is detected in the ventrally located hypoglossus nucleus (A). In the mutant pup, the probe that recognizes the disrupted transcript labels the DMN demonstrating the presence of these cells (B). Also, labeling with the markers ChAT (E and F) and Phox2a (E and F) appear normal in the Nurr1^{-/-} as compared to the Nurr1^{+/+}. In contrast to the wild-type DMN, Ret can not be detected in the Nurr1^{-/-} nucleus at this stage (G and H). Scale bar: 400 μm. 4V=fourth ventricle, DMN=dorsal motor nucleus, HyN=hypoglossal nucleus.

FIG. 8 depicts Nurr1 protein expression in coronal sections of developing brainstem. Localization at E10.5 (A and B), E13.5 (C) and E12.5 (D) of Nurr1 protein (A-C, red), HB9 (A, green), a marker for somatic motor neurons, and Islet 1 (B-D, green), a marker for both somatic and visceral motor neurons. At rhombomere 6-7 level, somatic motor neurons detected with an antibody to HB9 (green) do not express Nurr1 as no colocalization is detected (A). Nurr1 immuno-reactive cells (marked by arrows) are mainly detected dorsally of the HB9 reactive somatic motor neurons that will form the hypoglossal nucleus. Nurr1 does colocalize with Islet 1 (B; yellow cells marked with arrows) in cells negative for HB9 (compare A and B), i.e., visceral motor neurons. At E13.5, the visceral DMN has formed laterally of the fourth ventricle and is positive for Nurr1 whereas Islet 1 is almost completely down-regulated (C). In the Nurr1 mutant brainstem, the DMN is present as shown by Islet 1 immunoreactivity at E12.5 (D). Section is at slightly different angle from that in (C). Scale bars: A 65 μm (also applies for B), C 90 μm (also applies for D). 4V=fourth ventricle, DMN=dorsal motor nucleus, HyN=hypoglossal nucleus, Isl1=Islet 1.

FIG. 9 depicts Nurr1 and Ret mRNA expression in the brainstem. Serial coronal sections of E13.5 (A-D) and E16.5 (E-H) brain stems with in situ hybridization for Nurr1 (A, C, E, G) and Ret (B, D, F, H). DMN on right side (A-H) and 4V (C, D, G, H) encircled with dotted lines for ease of identification. Nurr1 mRNA is detected in the DMN at E13.5 (A) and weak but distinct expression of Ret mRNA is detected in the DMN and in the hypoglossal nucleus (B). In the Nurr1 mutant, the detection of the DMN is by the probe that recognizes the disrupted Nurr1 transcript (C). No Ret signal is detected in the Nurr1 mutant DMN, but can be seen in the hypoglossal nucleus (D). Similarly, at E16.5, the DMN is recognized by In situ hybridization for Nurr1 in the wild-type and mutant brainstem (E and G) whereas Ret mRNA can be detected in this nucleus in the wild-type (F) but not in the mutant (H). Scale bars: B 80 μm (applies for A-D), F 130 μm (applies for E-H). 4V=fourth ventricle, DMN=dorsal motor nucleus, HyN=hypoglossal nucleus.

FIG. 10 depicts analysis of the vagus nerve in the E15.5 and E12.5 embryo. (A-D) Horizontal sections at thoracic-lumbar level of the mouse E15.5 trunk and (E-I) whole mount x-gal stained E12.5 Nkx6.2^{+/-} Nurr1^{+/-} and Nkx6.2^{+/-}:Nurr1^{-/-} embryos. Labeling for the general neuronal marker pgp9.5 visualizes the vagus nerve in the Nurr1^{+/+} embryo (A) as well as the Nurr1^{-/-} (B) animal. To analyze efferent fibers only, sections from Nkx6.2^{+/+}:Nurr1^{+/+} and Nkx6.2^{+/-}:Nurr1^{-/-} were analyzed for β-gal immunoreactivity which gives a punctuate pattern representing a subset of the pgp9.5 positive fibers. (C and D). Side views (E and F) of E12.5 x-gal stained embryos show the vagus nerve extending from the cranium into the trunk. Close up of vagus nerve fibers (G and H) exiting in 11 fiber tracts from the cranial nuclei within the tissue. The fibers appear longer in the Nkx6.2^{+/-}:Nurr1^{-/-} brain (H) than in the Nkx6.2^{+/-}:Nurr1^{+/+} brain (G) as visualized by a full white arrow in the wild-type that is compensated with a dotted arrow in the mutant to represent the total length of the fibers. Scale bars: A-D 15 μm, E and F 100 μm, G and H 30 μm. 4V=fourth ventricle, X=tenth cranial nerve; the vagus nerve.

FIG. 11 depicts horizontal-trunk sections of vagus-nerve-target areas. Left panel (A, D, G, J, M, P) bronchi in the lungs, middle panel (B, E, H, K, N, Q) esophagus, right panel (C, F, I, L, O, R) intestines and at bottom, additional sections of esophagus (S-U). (A-F) pgp9.5 immunoreactivity in different target areas in the newborn wild-type (A, B, C) and Nurr1 mutant mouse (D, E and F), (G-L) AChE assay visualization of esterase activity in the same areas in the E18.5 wild-type (G, H, I) and Nurr1 mutant (J, K, L) embryo, (M-R) VAChT immunoreactivity in same areas of newborn wild-type (M, N, O) and Nurr1 −/− (P, Q, R) pups shows normal neuronal innervation of muscle in all areas and also of submucosa in esophagus and intestines. In addition to myenteric and submucosal innervation of the esophagus, staining was also detected in the mucosa epithelium in the wild-type pup (S and close up of epithelium in T), however, this was not detected to the same extent in the mutant epithelium (U). Scale bars: A (also applies for B, D, E), C (also F), M (also P), N (also Q), O (also R), S (also T and U) 20 μm, G (also H, J, K) and I (also L) 40 μm. mus=muscle, sub=submucosa, epi=epithelium, X=vagus nerve.

FIG. 12 demonstrates the effect of the non-naturally-occurring-RXR ligand, LG100268. As used herein, “LG100268” and “LG268” are used interchangeably. Primary ventral midbrain cells were treated with increasing concentrations of two different RXR receptor agonists (A) SR11237 and (B) LG268. These ligands produced a dose-dependent increase in the number of TH-immunoreactive (IR) cells. (C) The efficiency of these ligands was compared to that of glial-cell-line-derived neurotrophic factor (GDNF), which is a known trophic factor for dopamine neurons. LG268 is more potent than either SR11237 or GDNF.

FIG. 13 demonstrates that RAR ligands inhibit the neurotrophic effect of RXR-specific ligands. (A) 9-cis retinoic acid (9cisRA) is a ligand for both RAR and RXR, and activates RAR more efficiently than RXR. The effect with increasing concentrations 9cisRA on primary midbrain cultures was analyzed with and without the RAR specific antagonist R041-5253 (RO) (Apfel et al. (1992) Proc. Nat'l. Acad. Sci. USA 89 (15): 7129-7133; incorporated herein by reference in its entirety). LG268 was used as a control. 9cisRA alone does not affect the number of TH-IR cells neither positively nor negatively. The number of TH-IR neurons increase with increasing concentrations of 9cisRA when combined with RO. Blocking RAR signaling enables 9cisRA to promote dopamine-cell survival through 9cisRA activation of RXR. This result suggests that RAR activation blocks the neurotrophic effect of RXR ligands. (B) TTNPB is an RAR-specific agonist and does not increase the number of TH-IR cells. In the presence of LG268, TTNPB blocks the neurotrophic effect of this ligand. These data indicate that RAR activation counteracts the trophic activity of RXR-specific ligands.

FIG. 14 demonstrates that docosahexanoic acid (DHA) is an RXR ligand and promotes dopaminergic cell survival by activating RXR. (A) Increasing concentrations of DHA increased the number of TH-IR cells in a dose-dependent manner. (B) LG1208—a selective RXR antagonist-blocks the neurotrophic activity of DHA. (C) Both LG268 and DHA efficiently increased the number of dopamine neurons in the cultures (as quantified by TH-staining), but do not produce a general increase in the entire neuronal-cell population. NeuN antibodies detect cells expressing a general neuronal marker. Quantification of NeuN-positive cells demonstrates that the effect of RXR ligands in primary ventral mesencephalic cultures is specific to dopaminergic cells.

FIG. 15 demonstrates that LG268 increases the number of Nurr1-positive cells, but does not increase the number of cells that do not express Nurr1. RXR can form heterodimers with a large number of nuclear receptors, including Nurr1. The experiments detailed here demonstrate that the RXR/Nurr1 complex promotes cell survival. Several Nurr1-expressing tissues-detected using a Nurr1-specific antiserum—were treated with LG268. The selected tissues include the cortex (A and B) and the hippocampus (C and D). The number of Nurr1-positive cells (A) increased by 39% after addition of LG268, while the number of non-expressing cells (B) did not increase. In hippocampal cultures, LG268 (C) increased the number of Nurr1 -positive cells by 36%, while the number of non-expressing cells (D) did not increase. These results strongly suggest that Nurr1-RXR heterodimers are involved in promoting dopamine secreting cell survival. Moreover, the results suggest that dopamine secreting cells from the cortex and hippocampus that express Nurr1 are permissive to the neurotrophic effects of RXR ligands.

FIG. 16 demonstrates that Nurr1 is the dimerization partner of RXR that is necessary to mediate the neurotrophic effects of the RXR ligand LG268. Both Nurr1-positive cells (A) (Nurr1^{+/+}) and Nurr1-negative cells (B) (Nurr1^{-/-}) from cortical cells treated with LG268.

### SUMMARY OF THE INVENTION

The present invention provides methods of increasing the survival of dopamine secreting cells, comprising the steps of administering a retinoid X receptor (RXR) ligand to dopamine secreting cells, wherein the binding of the RXR ligand increases the survival of the dopamine secreting cells. In one embodiment, the dopamine secreting cells of the present methods are isolated from the basal ganglia. Preferably, the dopamine secreting cells are isolated from the striatum, globus pallidus, substantia innominate, ventral pallidum, nucleus basalis of Meynert, ventral tegmental area, and the subthalmic nucleus. More preferably, the dopamine secreting cells are isolated from the substantia nigra.

The present invention also provides methods of increasing the survival of dopamine secreting cells, comprising the steps of administering a retinoid X receptor (RXR) ligand and an RAR antagonist to dopamine secreting cells, wherein the binding of the RXR ligand increases the survival of the dopamine secreting cells. In one embodiment, the dopamine secreting cells of the present methods are isolated from the basal ganglia. Preferably, the dopamine secreting cells are isolated from the striatum, globus pallidus, substantia innominate, ventral pallidum, nucleus basalis of Meynert, ventral tegmental area, and the subthalmic nucleus. More preferably, the dopamine secreting cells are isolated from the substantia nigra. In one embodiment, the RAR antagonist is RO41-5253 (RO). As used herein, RO41-5253 and RO415253 are used interchangeably.

The present invention also provides methods of increasing the survival of dopamine secreting cells, comprising the steps of administering RXR ligand to dopamine secreting cells, wherein the binding of the RXR ligand increases the survival of the dopamine secreting cells. Preferably, the RXR ligand is an agonist. More preferably, the RXR ligand activates RXR and the RXR-dependent-response pathway mediates the increased survival of the dopamine secreting cells. In one embodiment, the RXR ligand is: 1) isolated from ventral embryonic midbrain cells; 2) contained in conditioned media produced by incubating embryonic ventral midbrain explants in culture media; 3) a naturally-occurring-RXR ligand that is endogenous to the neuronal cells to which the RXR ligand is administered; 4) a naturally-occurring RXR ligand that is not endogenous to the neuronal cells to which the RXR ligands is administered; or 5) a non-naturally-occurring-RXR ligand or RXR-ligand analog. Preferably, the naturally-occurring-RXR ligand is 9-cis retinoic acid or docosahexaenoic acid.

The present invention also provides methods of increasing the survival of dopamine secreting cells, comprising the steps of administering RXR ligand and an RAR antagonist to dopamine secreting cells, wherein the binding of the RXR ligand increases the survival of the dopamine secreting cells. In one embodiment, the RAR antagonist is RO41-5253 (RO). Other RAR antagonists contemplated by this invention include RO-61-8431 (Rincon et al. (2002) J. Exp. Zool. 292 (22): 435-443; incorporated herein by reference in its entirety); RAR-beta-selective antagonists, including LE135, LE540 and LE550 (Li et al. (1999) J. Biol. Chem. 274 (22): 15360-15366; incorporated herein by reference in its entirety); RAR-pan antagonists, including AGN194310 (Hammond et al. (2001) Br. J. Cancer 271 (21): 12209-12212; incorporated herein by reference in its entirety), AGN193109 (Agarwal et al. (1996) J. Biol. Chem. 271 (21): 12209-12212; incorporated herein by reference in its entirety), and BMS-189453 (Schulze et al. (2001) Toxicol. Sci. 59 (2): 297-308; incorporated herein by reference in its entirety); RAR-beta and RAR-gamma-selective antagonists, including AGN194431 (Hammond et al. (2001) Br. J. Cancer 271 (21): 12209-12212; incorporated herein by reference in its entirety); and RAR-alpha selective antagonists, including AGN194301 (Hammond et al. (2001) Br. J. Cancer 271 (21): 12209-12212; incorporated herein by reference in its entirety). Preferably, the RXR ligand is an agonist. More preferably, the RXR ligand activates RXR and the RXR-dependent-response pathway mediates the increased survival of the dopamine secreting cells. In one embodiment, the RXR ligand is: 1) isolated from ventral embryonic midbrain cells; 2) contained in conditioned media produced by incubating embryonic ventral midbrain explants in culture media; 3) a naturally-occurring-RXR ligand that is endogenous to the neuronal cells to which the RXR ligand is administered; 4) a naturally-occurring- RXR ligand that is not endogenous to the neuronal cells to which the RXR ligands is administered; or 5) a non-naturally-occurring-RXR ligand or RXR-ligand analog. Preferably, the naturally-occurring-RXR ligand is 9-cis retinoic acid or docosahexaenoic acid.

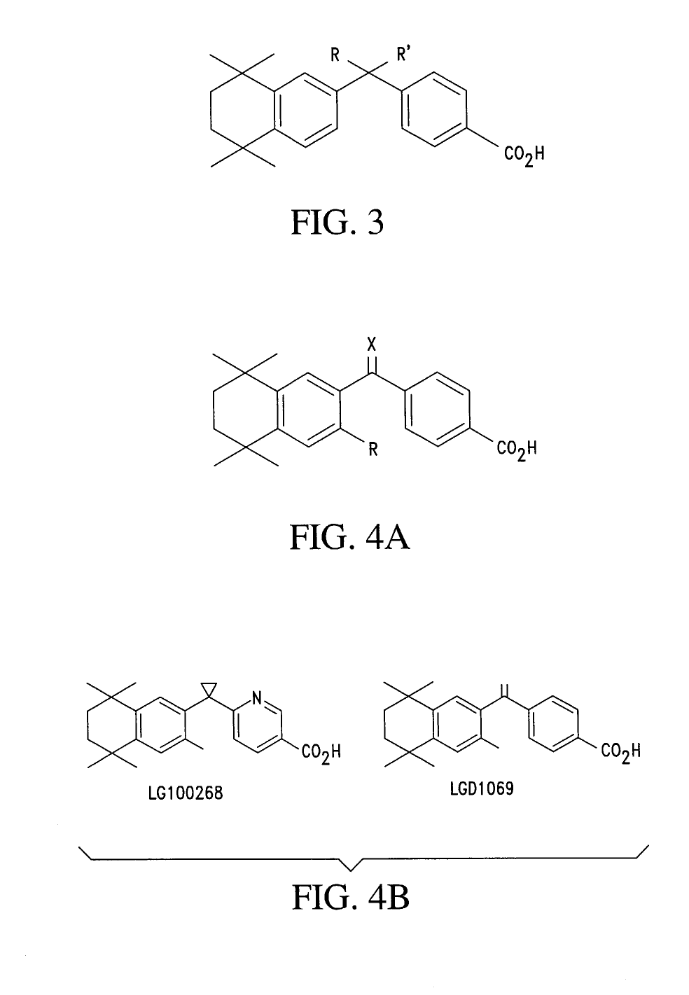

In another embodiment, the RXR ligand is a non-naturally-occurring ligand or an RXR-ligand analog. Preferably, the non-naturally-occurring-RXR ligand or RXR-ligand analog selectively activates RXR-mediated-response pathways. More preferably, the non-naturally-occurring-RXR ligand or RXR-ligand analog activates RXR and RXR-mediated-response pathways, but does not substantially activate RXR-RAR heterodimers and RAR-dependent-response pathways. Most preferably, the non-naturally-occurring-RXR ligand or RXR-ligand analog is selected from the group consisting of: LG1069 (Boehm et al. (1994) J. Med. Chem. 37: 2930-2941; Mukherjee et al. (1997) Nature 386: 407-410; both incorporated herein by reference), LG100268 (Boehm et al. (1995) J. Med. Chem. 38: 3146-3155; Mukherjee et al. (1997) Nature 386: 407-410; both incorporated herein by reference), SR11203, SR11217, SR11234, SR11235, SR11236 and SR11237 (Lehmann et al. (1992) Science 258:1944-1946; incorporated herein by reference; see also, FIG. 3).

The present invention provides methods for increasing the survival of dopamine secreting cells, in vitro and in vivo. In one embodiment of the invention, an RXR ligand is administered to a subject in need thereof, in an amount sufficient to bind RXR and increase survival of dopamine secreting cells. In a preferred embodiment, the subject has a neurodegenerative disease that is characterized by the loss of dopamine secreting cells. In a more preferred embodiment, the neurological disease is characterized by the loss of dopamine-secreting cells. In the most preferred embodiment, the neurodegenerative disease is Parkinson's disease. Preferably, the RXR ligand is: 1) isolated from ventral embryonic midbrain cells; 2) contained in conditioned media produced by incubating embryonic ventral midbrain explants in culture media; 3) a naturally-occurring-RXR ligand that is endogenous to the neuronal cells to which the RXR ligand is administered; 4) a naturally-occurring-RXR ligand that is not endogenous to the neuronal cells to which the RXR ligands is administered; or 5) a non-naturally-occurring-RXR ligand or RXR-ligand analog. Preferably, the naturally-occurring-RXR ligand is 9-cis retinoic acid or docosahexaenoic acid. Preferably, the non-naturally-occurring-RXR ligand or RXR-ligand analog selectively activates RXR-mediated response pathways. More preferably, the non-naturally-occurring-RXR ligand or RXR-ligand analog activates RXR and RXR-mediated response pathways, but does not substantially activate RXR-RAR heterodimers and RAR-dependent-response pathways. Most preferably, the non-naturally-occurring-RXR ligand or RXR-ligand analog is selected from the group consisting of: LG1069 (Boehm et al. (1994) J. Med. Chem. 37: 2930-2941; Mukherjee et al. (1997) Nature 386: 407-410; both incorporated herein by reference), LG100268 (Boehm et al. (1995) J. Med. Chem. 38: 3146-3155; Mukherjee et al. (1997) Nature 386: 407-410; both incorporated herein by reference), SR11203, SR11217, SR11234, SR11235, SR11236 and SR11237 (Lehmann et al. (1992) Science 258:1944-1946; incorporated herein by reference; see also, FIG. 3). All the methods of increasing the survival of dopamine secreting cells in a subject in need thereof, can further comprise administration of an RAR antagonist, either simultaneously or before or after administration of the RXR ligand. Preferably, RAR antagonist may be any molecule that binds to RAR and prevents activation of RAR, activation of any RAR-mediated signaling pathway, or any biological response mediated by an RAR ligand or RAR agonist. More preferably the RAR antagonist is selected from the group consisting of: RO41-5253 (RO), RO-61-8431, LE135, LE540, LE550, AGN194310, AGN193109, BMS-189453, AGN194431, and AGN194301.

The present invention provides methods of increasing the survival of dopamine secreting cells, in vitro, by activating RXR and RXR-dependent pathways that increase the survival of treated cells. In one embodiment, the dopamine secreting cells treated in vitro are transplanted into a subject in need thereof, using methods known in the art. Preferably, the subject has a neurodegenerative disease. More preferably, the subject has Parkinson's disease.

The present invention also provides methods of treating subjects with neurodegenerative disease, comprising the treatment of dopamine secreting cells with RXR ligands in vitro, followed by transplantation into a subject in need thereof. Preferably, the dopamine secreting cells are derived from the retina, olfactory bulb, hypothalamus, dorsal motor nucleus, nucleus tractus solitarious, periaqueductal gray matter, ventral tegmentum, or substantia nigra.

The transplanted dopamine secreting cells used in the methods of the present invention are preferably treated with a naturally-occurring RXR ligand. Preferably, the naturally-occurring RXR ligand is 9-cis retinoic acid or docosahexaenoic acid. Other embodiments of the present invention include administration of an RXR ligand that is: 1) isolated from ventral embryonic midbrain cells; 2) contained in conditioned media produced by incubating embryonic ventral midbrain explants in culture media; 3) a naturally-occurring RXR ligand that is endogenous to the neuronal cells to which the RXR ligand is administered; 4) a naturally-occurring RXR ligand that is not endogenous to the neuronal cells to which the RXR ligands is administered; or 5) a non-naturally-occurring-RXR ligand or RXR-ligand analog.

In one embodiment, the non-naturally-occurring-RXR ligand or RXR-ligand analog activates RXR and RXR-mediated response pathways, but does not substantially activate RXR-RAR heterodimers and RAR-dependent-response pathways. Preferably, the non-naturally-occurring-RXR ligand or RXR-ligand analogs are selected from the group consisting of: LG1069 (Boehm et al. (1994) J. Med. Chem. 37: 2930-2941; Mukherjee et al. (1997) Nature 386: 407-410; both incorporated herein by reference) LG100268 (Boehm et al. (1995) J. Med. Chem. 38: 3146-3155; Mukherjee et al. (1997) Nature 386: 407-410; both incorporated herein by reference), SR11203, SR11217, SR11234, SR11235, SR11236 and SR11237 (Lehmann et al. (1992) Science 258:1944-1946; incorporated herein by reference; see also, FIG. 3).

The present invention also provides a method for identifying a gene that is under the transcriptional control of RXR, wherein the method comprises the steps of: 1) contacting a neuronal cell with an RXR ligand that activates RXR and an RXR-dependent-response pathway that mediates the increased survival of the dopamine secreting cells; and 2) identifying transcripts that are differentially expressed in the treated cells, as compared to transcripts present in untreated cells, wherein the differentially expressed transcripts in the treated cells are encoded by genes under the transcriptional control of RXR. In one embodiment, the method further comprises the step of isolating transcripts from the cells. In yet another embodiment, the method further comprises the additional steps of isolating transcripts from the treated cells and comparing those transcripts to transcripts isolated from neuronal cells that have not been treated with the ligand. The identity of the differentially transcribed gene may be determined by using methods well known in the art, such as differential display, array analysis or by serial analysis of gene expression (SAGE).

The dopamine secreting cells of the present invention are preferably isolated from the retina, olfactory bulb, hypothalamus, dorsal motor nucleus, nucleus tractus solitarious, periaqueductal gray matter, ventral tegmentum, or substantia nigra. More preferably, the dopamine secreting cells are dopamine-secreting cells. The RXR ligand may be a naturally-occurring ligand. Preferably, the naturally-occurring RXR ligand is 9-cis retinoic acid or docosahexaenoic acid. Other embodiments of the present invention include administration of an RXR ligand that is: 1) isolated from ventral embryonic midbrain cells; 2) contained in conditioned media produced by incubating embryonic ventral midbrain explants in culture media; 3) a naturally-occurring RXR ligand that is endogenous to the neuronal cells to which the RXR ligand is administered; 4) a naturally-occurring RXR ligand that is not endogenous to the neuronal cells to which the RXR ligands is administered; or 5) a non-naturally-occurring-RXR ligand or RXR-ligand analog.

Preferably, the non-naturally-occurring-RXR ligand or an RXR-ligand analog selectively activates RXR-mediated response pathways. More preferably, the non-naturally-occurring-RXR ligand or RXR-ligand analog activates RXR and RXR-mediated response pathways, but do not substantially activate RXR-RAR heterodimers and RAR-dependent-response pathways. Most preferably, the non-naturally-occurring-RXR ligand or RXR-ligand analog is selected from the group consisting of: LG1069 (Boehm et al. (1994) J. Med. Chem. 37: 2930-2941; Mukherjee et al. (1997) Nature 386: 407-410; both incorporated herein by reference), LG100268 (Boehm et al. (1995) J. Med. Chem. 38: 3146-3155; Mukherjee et al. (1997) Nature 386: 407-410; both incorporated herein by reference), SR11203, SR11217, SR11234, SR11235, SR11236 and SR11237 (Lehmann et al. (1992) Science 258:1944-1946; incorporated herein by reference; see also, FIG. 3).

The present invention provides methods for identifying an agent that stimulates Nurr1, wherein the method comprises the steps of: 1) contacting a cell with an agent and 2) measuring Ret-gene expression; wherein an increase in Ret expression relative to an untreated cell indicates that the agent stimulates Nurr1 expression. Preferably, the cell is a neuronal cell, and more preferably a dopamine-secreting cell. In another embodiment, the cell is Nurr1^{-/-} and is transformed or transfected with a vector encoding Nurr1. Preferably, the cell is transformed or transfected with a vector comprising the Nurr1 gene and a vector comprising the Ret gene. The transfected or transformed cells, prior to transfection or transformation, may be Nurr1^{-/-} or Ret^{-/-}, or Nurr1^{-/-} and Ret^{-/-}. As used herein, “Nurr1^{-/-} cell” refers to a cell that lacks a functional copy of the Nurr1 gene, and consequently, does not produce Nurr1 protein. As used herein, “Ret^{-/-} cell” refers to a cell that lacks a functional copy of the Ret gene, and consequently, does not produce Nurr1 protein. Ret-gene expression may be measured by any method routine in the art, e.g., Northern blot, quantitative PCR, differential display, and array analysis.

The present invention provides methods for identifying an agent that regulates Nurr1 comprising contacting a cell expressing Nurr1 and Ret with an agent to be assayed and measuring Ret expression, wherein a difference in Ret expression compared to a control cell not contacted with said agent indicates the agent regulates Nurr1. In the methods of this invention, an increase in Ret expression indicates that the agent stimulates Nurr1 and a decrease in Ret expression indicates the agent inhibits Nurr1. Preferably, the cell is transformed or transfected with a vector comprising the Nurr1 gene and a vector comprising the Ret gene. The transfected or transformed cells, prior to transfection or transformation, may be Nurr1^{-/-} or Ret^{-/-}, or Nurr1^{-/-} and Ret^{-/-}.

The present invention also provides methods of increasing the expression of Ret in dopamine secreting cells, wherein the method comprises the step of administering an agent to a subject in need thereof, wherein the agent increases Ret expression. The dopamine secreting cells may be obtained from the basal ganglia, including, for example, dopamine secreting cells from the striatum, globus pallidus, substantia innominate, ventral pallidum, nucleus basalis of Meynert, ventral tegmental area, the subthalmic nucleus, and the substantia nigra.

Preferably, the agent is either a Nurr1 ligand or an RXR ligand, and more preferably an RXR ligand that selectively activates RXR. RXR ligands useful for this method include both naturally-occurring and non-naturally-occurring ligands. Preferred naturally-occurring RXR ligands are isolated from ventral embryonic midbrain cells. Most preferably, the naturally-occurring RXR ligand is 9-cis retinoic acid or docosahexanoic acid. Preferred non-naturally-occurring RXR ligands are selected from the group consisting of: LG1069, LG100268, SR11203, SR11217, SR11234, SR11235, SR11236 and SR11237. The present invention also provides methods of increasing the expression of Ret in the dopamine secreting cells of a subject that has a neurodegenerative disease. Preferably, the neurodegenerative disease is characterized by loss of dopamine-secreting cells. Most preferably, the neurodegenerative disease is Parkinson's disease.

The present invention also provides methods of increasing the expression of Ret in dopamine secreting cells, wherein the method comprises the step of administering an agent to a subject in need thereof, wherein the agent activates Nurr1, thereby increasing the expression of Ret in dopamine secreting cells. The dopamine secreting cells may be obtained from the basal ganglia, including, for example, dopamine secreting cells from the striatum, globus pallidus, substantia innominate, ventral pallidum, nucleus basalis of Meynert, ventral tegmental area, the subthalmic nucleus, and the substantia nigra.

Preferably, the agent is either a Nurr1 ligand or an RXR ligand, and more preferably an RXR ligand that selectively activates RXR. RXR ligands useful for this method include both naturally-occurring and non-naturally-occurring ligands. Preferred naturally-occurring RXR ligands are isolated from ventral embryonic midbrain cells. Most preferably, the naturally-occurring RXR ligand is 9-cis retinoic acid or docosahexanoic acid. Preferred non-naturally-occurring RXR ligands are selected from the group consisting of: LG1069, LG100268, SR11203, SR11217, SR11234, SR11235, SR11236 and SR11237. The present invention provides methods of increasing the expression of Ret in the dopamine secreting cells of a subject that has a neurodegenerative disease. Preferably, the neurodegenerative disease is characterized by loss of dopamine-secreting cells. Most preferably, the neurodegenerative disease is Parkinson's disease.

The present invention provides a use of an RXR ligand in the manufacture of a medicament for treatment of a condition associated with loss of dopamine secreting cells. Preferably, the RXR ligand selectively activates RXR. In one embodiment, the RXR ligand is a naturally-occurring ligand. These naturally-occurring RXR ligands are preferably isolated from ventral embryonic midbrain cells. More preferably the naturally-occurring ligand is 9-cis retinoic acid or docosahexaenoic acid. In another embodiment, the RXR ligand is a non-naturally occurring RXR ligand. Preferably, the non-naturally occurring RXR ligand is selected from the group consisting of: LG1069, LG100268, SR11203, SR11217, SR11234, SR11235, SR11236 and SR11237.

The present invention provides a use of an RXR ligand and an RAR antagonist in the manufacture of a medicament for treatment of a condition associated with loss of dopamine secreting cells. Preferably, the RXR ligand selectively activates RXR. In one embodiment, the RXR ligand is a naturally-occurring ligand. These naturally-occurring RXR ligands are preferably isolated from ventral embryonic midbrain cells. More preferably the naturally-occurring ligand is 9-cis retinoic acid or docosahexaenoic acid. In another embodiment, the RXR ligand is a non-naturally occurring RXR ligand. Preferably, the non-naturally occurring RXR ligand is selected from the group consisting of: LG1069, LG100268, SR11203, SR11217, SR11234, SR11235, SR11236 and SR11237.

The present invention provides a use of an RXR ligand in the manufacture of a medicament for treatment of a patient suffering from a condition associated with loss of dopamine secreting cells, wherein said patient has been treated with an RAR antagonist. Preferably, the RXR ligand selectively activates RXR. In one embodiment, the RXR ligand is a naturally-occurring ligand. These naturally-occurring RXR ligands are preferably isolated from ventral embryonic midbrain cells. More preferably the naturally-occurring ligand is 9-cis retinoic acid or docosahexaenoic acid. In another embodiment, the RXR ligand is a non-naturally occurring RXR ligand. Preferably, the non-naturally occurring RXR ligand is selected from the group consisting of: LG1069, LG100268, SR11203, SR11217, SR11234, SR11235, SR11236 and SR11237.

The present invention provides a use of an RAR antagonist for the manufacture of a medicament to enhance the effect of an RXR ligand administered subsequently in the treatment of a patient suffering from a condition associated with loss of dopamine secreting cells. Preferably, the RXR ligand selectively activates RXR. In one embodiment, the RXR ligand is a naturally-occurring ligand. These naturally-occurring RXR ligands are preferably isolated from ventral embryonic midbrain cells. More preferably the naturally-occurring ligand is 9-cis retinoic acid or docosahexaenoic acid. In another embodiment, the RXR ligand is a non-naturally occurring RXR ligand. Preferably, the non-naturally occurring RXR ligand is selected from the group consisting of: LG1069, LG100268, SR11203, SR11217, SR11234, SR11235, SR11236 and SR11237.

The present invention provides an RXR ligand for use in a method of the treatment of a condition associated with the loss of dopamine secreting cells. Preferably, the RXR ligand selectively activates RXR. In one embodiment, the RXR ligand is a naturally-occurring ligand. These naturally-occurring RXR ligands are preferably isolated from ventral embryonic midbrain cells. More preferably the naturally-occurring ligand is 9-cis retinoic acid or docosahexaenoic acid. In another embodiment, the RXR ligand is a non-naturally occurring RXR ligand. Preferably, the non-naturally occurring RXR ligand is selected from the group consisting of: LG1069, LG100268, SR11203, SR11217, SR11234, SR11235, SR11236 and SR11237.

The present invention provides an RXR ligand and an RAR agonist for use in a method of the treatment of a condition associated with the loss of dopamine secreting cells. Preferably, the RXR ligand selectively activates RXR. In one embodiment, the RXR ligand is a naturally-occurring ligand. These naturally-occurring RXR ligands are preferably isolated from ventral embryonic midbrain cells. More preferably the naturally-occurring ligand is 9-cis retinoic acid or docosahexaenoic acid. In another embodiment, the RXR ligand is a non-naturally occurring RXR ligand. Preferably, the non-naturally occurring RXR ligand is selected from the group consisting of: LG1069, LG100268, SR11203, SR11217, SR11234, SR11235, SR11236 and SR11237. Preferably the RAR antagonist is selected from the group consisting of: R041-5253 (RO), RO-61-8431, LE135, LE540, LE550, AGN194310, AGN193109, BMS-189453, AGN194431, and AGN194301.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### DETAILED DESCRIPTION OF THE INVENTION

### 1. RXR Ligand-Treatment of Dopamine Secreting Cells, In Vitro

The present invention is based on the surprising finding that the binding of ligands to RXR increases the survival of dopamine secreting cells. The present invention provides methods of increasing the survival of dopamine secreting cells comprising the steps of administering RXR ligand to dopamine secreting cells, in vitro, wherein the binding of the ligand to RXR increases the survival of the dopamine secreting cells.

As used herein, “RXR” refers to any retinoid X receptor. Mammalian RXR forms, especially human, are preferred, but other forms (i.e., simian, or murine, e.g., mouse, rat, hamster or other rodent forms) may also be used. Included are the highly related RXR receptors: RXRα, RXRβ and RXRγ (NR2B1, NR2B2, and NR2B3, respectively). As used herein, “RXR” also refers to all homodimeric and all heterodimeric forms of RXR. “RXR” may be a full-size RXR or a truncated form of an RXR molecule that includes the ligand-binding domain.

As used herein, “RAR antagonist” refers to any molecule that binds to RAR and prevents activation of RAR, activation of any RAR-mediated signaling pathway, or any biological response mediated by an RAR ligand or RAR agonist. RAR antagonists are well-known in the art. (See e.g., Chambon et al. (2000) U.S. Pat. No. 6,130, 230, columns 8-12; Bollag et al. (2000) U.S. Pat. No. 6,133,309; Basset et al. (2001) U.S. Pat. No. 6,184,256; each incorporated herein by reference). RAR antagonists include RO41-5253 (RO), RO-61-8431 (Rincon et al. (2002) J. Exp. Zool. 292 (22): 435-443; incorporated herein by reference in its entirety); RAR-beta-selective antagonists, including LE135, LE540 and LE550 (Li et al. (1999) J. Biol. Chem. 274 (22): 15360-15366; incorporated herein by reference in its entirety); RAR-pan antagonists, including AGN194310 (Hammond et al. (2001) Br. J. Cancer 271 (21): 12209-12212; incorporated herein by reference in its entirety), AGN193109 (Agarwal et al. (1996) J. Biol. Chem. 271 (21): 12209-12212; incorporated herein by reference in its entirety), and BMS-189453 (Schulze et al. (2001) Toxicol. Sci. 59 (2): 297-308; incorporated herein by reference in its entirety); RAR-beta and RAR-gamma-selective antagonists, including AGN194431 (Hammond et al. (2001) Br. J. Cancer 271 (21): 12209-12212; incorporated herein by reference in its entirety); and RAR-alpha selective antagonists, including AGN194301 (Hammond et al. (2001) Br. J. Cancer 271 (21): 12209-12212; incorporated herein by reference in its entirety).

As used herein, “RXR ligand” refers to any molecule, agonist or antagonist, that binds to RXR. Preferably, the RXR ligand is an agonist. In another embodiment, the RXR ligand is a naturally-occurring RXR ligand, such as 9-cis retinoic acid and docosahexaenoic acid. As used herein, “naturally-occurring” refers to a compound that is produced and found in nature. As used herein, “non-naturally-occurring” refers to compounds produced synthetically and that are not produced or found in nature. Preferably, the RXR ligands are retinoic acid analogs. In another embodiment, RXR ligands are isolated from ventral embryonic midbrain cells, or contained in conditioned media produced by incubating embryonic ventral midbrain explants in culture media. Other preferred RXR ligands include: i.e., naturally-occurring-RXR ligands endogenous to the neuronal cells to which the RXR ligands are administered; naturally-occurring RXR ligands that are not endogenous to the dopamine-secreting cells to which the RXR ligand is administered; and a non-naturally-occurring-RXR ligand or RXR-ligand analog. As used herein, “endogenous RXR ligands” refers to those RXR ligands that occur naturally in the cells to which the RXR ligands are administered. For example, a non-naturally-occurring-RXR ligand is not endogenous to any cell.

A more preferred RXR ligand is selected from the class of RXR ligands and RXR-ligand analogs that selectively activate RXR-mediated response pathways. As used herein, “selectively activates RXR” refers to an RXR ligand that activates RXR and RXR-mediated response pathways, but does not substantially activate RXR-RAR heterodimers and RAR-dependent-response pathways. These RXR ligands include naturally-occurring analogs that are either synthesized or isolated from a biological sample, or non-naturally-occurring-RXR ligands and RXR-ligand analogs, such as retinoids, retinoid-like compounds, retinoid heterocycles, and retinobenzoic acid derivatives, including LG1069 (Starrett, Jr. et al. U.S. Pat. No. 5,559,248; Boehm et al. (1994) J. Med. Chem. 37: 2930-2941; Mukherjee et al. (1997) Nature 386: 407-410; all incorporated herein by reference), LG100268 (Starrett, Jr. et al. U.S. Pat. No. 5,559,248; Boehm et al. (1995) J. Med. Chem. 38: 3146-3155; Mukherjee et al. (1997) Nature 386: 407-410; all incorporated herein by reference), SR11203, SR11217, SR11234, SR11235, SR11236 and SR11237 (Starrett, Jr. et al. U.S. Pat. No. 5,559,248; Lehmann et al. (1992) Science 258:1944-1946; both incorporated herein by reference; see also, FIG. 3).

A number of methods for screening candidate RAR antagonists, RXR ligands, and RXR-ligand analogs, including those agonists and antagonists generated by rational design or computer modeling, are well-known in the art. These methods allow one of ordinary skill to determine if a compound is useful in the methods of the present invention. Numerous reporter assays that were developed to identify RAR agonists and antagonists are also applicable to screening for RXR ligands and RXR-ligand analogs. (Chen et al. (1995) EMBO J. 14(6):1187-1197; Lehmann et al. (1992) Science 258:1944-1946; both incorporated herein by reference). Although the referenced reporter assays utilize the luciferase or thymidine kinase genes, other reporters, such as Neo, CAT, β-galactosidase or Green Fluorescent Protein, are well known in the art and may be used to identify RXR ligands and RXR-ligand analogs.

Other routine assays have been used to screen compounds that affect the function of other nuclear receptors, such as steroid receptors. (Bollag et al. U.S. Pat. No. 6,133,309; Basset et al. U.S. Pat. No. 6,184256; Chambon et al. U.S. Pat. No. 6,130,230; all incorporated herein by reference). A transient expression/gel retardation system has been used to study the effects of the synthetic steroids RU486 and R5020 on glucocorticoid and progesterone receptor function (Neyer et al. (1990) EMBO J. 9(12): 3923-3932; incorporated herein by reference). Similar assays have been used to show that tamoxifen competitively inhibits estradiol-induced ERAP 160 binding to the estrogen receptor. (Halachimi et al. (1994) Science 264: 1455-1458; incorporated herein by reference). Since the RAR and RXR receptors are structurally similar to other nuclear receptors, such as the steroid receptors, routine assays of this type may be useful in assessing the activities of these compounds on RAR or RXR receptors.

Other methods for identifying RXR ligands and RXR-ligand analogs, that are routine in the art may also be used. One skilled in the art will be able to determine which compounds are agonists or antagonists.

As used herein, “increases the survival of the dopamine secreting cells” refers to the effect of an RXR ligand on cell number, in culture, that is not attributable to cell proliferation. Survival is assayed by determining the difference in the number of surviving dopamine secreting cells following incubation with an RXR ligand, in culture, as compared to a control, i.e., the number of cells in culture following incubation with an RAR ligand (i.e., TTNPB), all-trans retinoic acid, or in the absence of an RXR ligand. The number of cells present in culture may be determined by numerous methods that are well known in the art. For example, these methods include manual and automated cell counting, total protein determinations, immunoassay, and the like. Similarly, cellular proliferation may be determined by numerous methods that are well known in the art. For example, these methods include bromodeoxyuridine (BrdU) labeling of DNA in actively replicating cells, and the like.

Dissociated neuronal cells may be placed into any known culture medium capable of supporting cell growth, including HEM, DMEM, RPMI, F-12, and the like. The culture medium may contain any supplement that is required for cellular nutrition, such as glutamine and other amino acids, vitamins, minerals and useful proteins such as transferrin and the like. Medium may also contain antibiotics, such as penicillin, streptomycin, gentamicin and the like, to prevent contamination with yeast, bacteria and fungi. In some cases, the medium may contain serum derived from bovine, equine, chicken and the like.

As used herein, “neuronal cell” refers to cells of the central-nervous system, including neurons, astrocytes, oligodendrocytes and the like. In a preferred embodiment, the neuronal cells of the present invention are cells derived from the basal ganglia, more preferably from the striatum, globus pallidus, substantia innominate, ventral pallidum, nucleus basalis of Meynert, ventral tegmental area, and the subthalmic nucleus. In a more preferred embodiment, dopamine secreting cells are those cells that are derived from the substantia nigra. In a most preferred embodiment, the neuronal cells of the present invention are dopamine-secreting cells.

The term “dopamine-secreting cells” refers to the individual cells and tissue from regions of the central-nervous system that are known, in the mature state, to contain significant numbers of dopaminergic cell bodies. Dopamine-secreting cells are found in regions of the retina, olfactory bulb, hypothalamus, dorsal motor nucleus, nucleus tractus solitarious, periaqueductal gray matter, ventral tegmentum, and sub stantia nigra. Tyro sine hydroxylase (TH), a dopamine-bio synthetic enzyme, is routinely used in the art as a marker for dopamine-secreting neurons. In addition, dopamine-secreting cells may be identified, for example, by the presence of dopamine decarboxylase, and/or the absence of dopamine betahydroxylase within the cells, or by other cellular markers that are well known in the art.

Primary cell cultures may be plated at a density that is preferably in the range of about 10² to 10⁷ cells/ml, and more preferably at a density of about 10⁶ cells/ml. The cells may be grown in any suitable container, such as a tissue culture flask, well, or petri dish. The container may or may not have a surface onto which cells can adhere. In cases where it is desirable that cells adhere, it is generally necessary to treat them with a substance that provides an ionically charged surface such as poly-D-lysine, poly-L-ornithine, Matrigel, laminin, fibronectin, and other surfaces known to induce cell attachment. Cells are capable of adhering to certain plastics. For example, a poly-L-ornithine-treated culture container provides a particularly suitable surface onto which cells can adhere. Glass substrates or untreated plastic tissue culture substrates can be used when adherence is not desired. The cells can be cocultured on a feeder-layer bed of any type or combination of types of cells. A feeder bed of other neural cells such as neurons, astrocytes or oligodendrocytes is preferred.

The cultures are maintained at or near physiological conditions. The pH is preferably between pH 6 to 8, more preferably between about pH 7.2 to 7.6, and most preferably at a pH of about 7.4. Cells are incubated preferably between 30 to 40° C., more preferably between about 32 to 38° C., and most preferably between about 35 to 37.5° C. The cells should be maintained in about 5% CO₂, 95% O₂, and 100% humidity. Culture conditions, however, may be varied. For example, the addition of 1% fetal bovine serum (FBS) increases the number of dopamine-secreting cells detected after a 24-hour coculture on a glial-cell-feeder layer. FBS (1%) also increases the numbers of dopamine-secreting cells when the cells are grown in undefined culture medium, in the absence of a feeder layer.

### 2. Treatment of Neurodegenerative Disease-RXR Ligand

The present invention provides methods of increasing the survival of dopamine secreting cells, in vivo. In one embodiment of the invention, an RXR ligand is administered to a subject in need thereof, in an amount sufficient to increase the survival of the dopamine secreting cells. Preferably, the RXR ligand is administered in the form of a pharmaceutical composition, wherein the pharmaceutical composition comprises an RXR ligand and a pharmaceutically acceptable carrier. As used herein, “pharmaceutically acceptable carrier” refers to any carrier, solvent, diluent, vehicle, excipient, adjuvant, additive, preservative, and the like, including any combination thereof, that is routinely used in the art.

Physiological saline solution, for example, is a preferred carrier, but other pharmaceutically acceptable carriers, such as artificial CSF, are also contemplated by the present invention. The primary solvent in such a carrier may be either aqueous or non-aqueous. The carrier may contain other pharmaceutically acceptable excipients for modifying or maintaining pH, osmolarity, viscosity, clarity, color, sterility, stability, rate of dissolution, and/or odor. Similarly, the carrier may contain still other pharmaceutically acceptable excipients for modifying or maintaining the stability, rate of dissolution, release, or absorption or penetration across the blood-brain barrier.

RXR ligands may be administered orally, topically, parenterally, rectally or by inhalation spray in dosage unit formulations that contain conventional non-toxic pharmaceutically acceptable carriers, adjuvants and vehicles. As used herein, “parenterally” refers to subcutaneous, intravenous, intramuscular, intrasternal, intrathecal, and intracerebral injection, including infusion techniques.

RXR ligands may be administered parenterally in a sterile medium. The RXR ligand, depending on the vehicle and concentration used, may be suspended or dissolved in the vehicle. Advantageously, adjuvants such as local anesthetics, preservatives and buffering agents can be dissolved in the vehicle. In a preferred embodiment, the therapeutic or pharmaceutical composition of the present invention is administered parenterally by injection or directly into the cerebral spinal fluid (CSF) by continuous infusion from an implanted pump. The most preferred route of parenteral administration of the pharmaceutical compositions of RXR ligands is subcutaneous, intramuscular, intrathecal or intracerebral. Other embodiments of the present invention encompass administration of the composition in combination with one or more agents that promote penetration of RXR ligands across the blood-brain barrier, and/or slow-release of the active ingredient(s). Such excipients include those substances usually and customarily used to formulate dosages for parenteral administration in either unit dose or multi-dose form or for direct infusion into the CSF by continuous or periodic infusion from an implanted pump.

The desired or optimal dose of RXR ligand may be obtained by parenteral administration that is repeated daily, more frequently, or less frequently. RXR ligand may also be infused continuously or periodically from an implanted pump. The frequency of dosing will depend on the pharmacokinetic parameters of the specific RXR ligand in the formulation and the route of administration.

In more preferred embodiments, the therapeutic or pharmaceutical compositions are administered as orally active formulations, inhalant spray or suppositories. The pharmaceutical compositions containing RXR ligands may be in a form suitable for oral use, for example, as tablets, troches, lozenges, aqueous or oily suspensions, dispersible powders or granules, emulsions, hard or soft capsules, syrups or elixirs.

Active ingredient may be combined with the carrier materials in an amount to produce a single dosage form. The amount of the active ingredient will vary, depending upon the identity of the specific RXR ligand, the host treated, and the particular mode of administration. Preferably, the amount of RXR ligand is sufficient to promote survival of dopamine secreting cells, particularly dopamine-secreting cells. The dose of RXR ligand administered to a subject is preferably between 0.1 to 100 mg/kg, more preferably between 0.2 to 50 mg/kg and most preferably, between 0.5 to 25 mg/kg. For example, the non-naturally-occurring-RXR ligand, LG100268, has been administered to mice at a dose of 20 mg/kg. (Mukherjee et al. (1997) Nature 386: 407-410). Dosage unit forms will generally contain between from about 1 mg to about 500 mg of RXR ligand. It will be understood by those skilled in the art, however, that specific dosage levels for specific patients will depend upon a variety of factors, including the activity of the specific RXR ligand utilized, the age, body weight, general health, sex, diet, time of administration, route of administration, and rate of excretion, drug combination and the severity of the particular disease undergoing therapy. Administration of the RXR ligands may require either one or multiple dosings.

Regardless of the manner of administration, however, the specific dose is calculated according to approximate body weight or body surface area of the patient. Further refinement of the dosing calculations necessary to optimize dosing for each of the contemplated formulations is routinely conducted by those of ordinary skill in the art without undue experimentation, especially in view of the dosage information and assays disclosed herein.

As used herein, “neurodegenerative” or “neuronal cell degeneration” refers to a process or disease process that leads to the death of neuronal cells of the central-nervous system, or the loss of neuronal cell function and/or other cells of the nervous system. In a preferred embodiment, the neurodegenerative disease is Parkinson's Disease. Other neurodegenerative diseases contemplated by the methods of the present inventions, including Alzheimer's Disease, Multiple Sclerosis, Huntington's Disease, Amylotrophic Lateral Sclerosis, and Parkinson's Disease, are associated with degeneration of neuronal cells, in specific locations of the central-nervous system, that lead to the loss of cell or tissue function.

Specifically, degeneration of the basal ganglia leads to cognitive and motor dysfunction, depending on the exact location. The basal ganglia consists of many separate regions, including the striatum (which consists of the caudate and putamen), the globus pallidus, the sub stantia nigra, sub stantia innominate, ventral pallidum, nucleus basalis of Meynert, ventral tegmental area and the subthalmic nucleus. For example, Alzheimer's disease is characterized by profound cellular degeneration of the forebrain and cerebral cortex, and localized degeneration of the nucleus basalis of Meynert. Huntington's Chorea is associated with the degeneration of neurons in the striatum, which produces involuntary jerking movements. Degeneration of the subthalmic nucleus is associated with athetosis (slow writhing movement). In Parkinson's disease, degeneration occurs in the substantia nigra par compacta.

As used herein, “a subject in need thereof” is a subject that displays clinical symptoms of neurodegeneration, or a subject with subclinical neurodegeneration that has, for example, an average annual 0.5-1.0% rate of decline of striatal-dopamine function, as determined by serial positron emission tomography (PET) or single emission computed tomography (SPECT). (Brooks (1998) Ann. Neurol. (suppl) 44: S10-S18; Bernheimer et al. (1973) J. Neurol. Sci. 20: 415-455; Fernley et al. (1991) Brain 114: 2283-2301; McGee et al. (1989) Ann. Neurol. 24: 574-576; each incorporated herein by reference).

### 3. Treatment of Neurodegenerative Disease-Transplantation

A preferred treatment of neurodegenerative disease prevents or reduces neuronal cell degeneration. Once damage has occurred, it would be preferable to replace the lost cells by implanting new dopamine secreting cells, derived from dopamine secreting cells that have been proliferated in culture.

It is well recognized that transplantation of neuronal cells offers a powerful treatment for neurodegenerative disorders. (Lindvall (1991) TINS 14(8): 376-383; incorporated herein by reference). The transplantation of neuronal cells into damaged neural tissue has the potential to repair and/or replace damaged circuits and provide a replacement source of neurotransmitters, thereby restoring neurological function. The absence of suitable cells for transplantation purposes, however, has prevented the full potential of this procedure from being met. As used herein, “suitable cells” are those cells that meet the following criteria: 1) cells that can be obtained in large numbers; 2) cells that can be proliferated, in vitro; 3) cells that are capable of surviving indefinitely, but stop growing after transplantation to the brain; 4) are nonimmunogenic, preferably obtained from a patient's own tissue; 5) are able to form normal neural connections and respond to neural physiological signals. (Bjorklund (1991) TINS 14(8): 319-322). Dopamine secreting cells that have been treated with RXR ligands meet the requirements of cells suitable for neural transplantation purposes. Furthermore, these dopamine secreting cells can be derived from the patient, a compatible tissue-typed donor, or an untyped donor wherein the cells are administered with local immunosuppression to minimize the potential of graft rejection.

The present invention provides methods of increasing the survival of dopamine secreting cells, in vitro, by treating cells with an RXR ligand that activates RXR and RXR-dependent pathways that increase the survival of treated cells. In a preferred embodiment, the treated dopamine secreting cells are transplanted into a subject in need thereof. In a preferred embodiment, the subject has a neurodegenerative disease, and in a more preferred embodiment, the subject has Parkinson's disease. This invention is based on the surprising discovery that dopamine secreting cells, following treatment with an RXR ligand, survive longer than untreated dopamine secreting cells even after RXR ligand-treatment is discontinued.

Dopamine secreting cells are the preferred cell for implantation. In a preferred embodiment, the dopamine secreting cells of the present invention are isolated from the basal ganglia, more preferably from the striatum, globus pallidus, substantia innominate, ventral pallidum, nucleus basalis of Meynert, ventral tegmental area, and the subthalmic nucleus. More preferably, dopamine secreting cells are those cells that are isolated from the substantia nigra. The dopamine secreting cells are preferably isolated from a non-tumor cell line, or from cells that have not been intentionally immortalized in order to induce proliferation. More preferably, the cells are isolated from a patient's own neural tissue.

Stem cells are a class of multipotent cell that function as a source of numerous cell types that replace cells lost by natural cell death, injury or disease. (Gage, F. H. and Christen, Y. (eds.) (1997) Isolation, Characterization, and Utilization of CNS Stem Cells—Research & Perspectives in Neurosciences, Springer-Verlag, Berlin Heidelberg; Fisher (1997) Neurobiol. Dis. 4: 1-22; Gage et al. (1995) Ann. Rev. Neurosci. 18: 159-92; Gage et al. (1995) Proc. Natl. Acad. Sci. USA 92: 11879-11883; Gage (1998) Nature 392 (supp): 18-24; Martinez-Serrano (1997) Trends Neurosci. 20: 530-538; McKay (1997) Science 276: 66-71; Reynolds et al. (1996) Develop. Biol. 175: 1-13; Snyder (1994) Curr. Opin. Neurobiol. 4: 742-751; Snyder et al. (1996) Curr. Opin. in Pediatr. 8: 558-568; Verma et al. (1997) Nature 389: 239-242; Whittemore et al. (1996) Molecular Neurobiology 12(1): 13-38; all incorporated herein by reference). As used herein, the phrase “stem cell” refers to a undifferentiated, multipotent cell that is capable of self maintenance (i.e., progeny are also stem cells), proliferates without limit, and produces progeny that can terminally differentiate into neurons and glia. As used herein, “neuronal stem cell” includes those undifferentiated cells that are derived from a multipotent neuronal stem cell, are committed to a particular path of differentiation (i.e., neurons, astrocytes and oligodendrocytes), and have limited proliferative and no self maintenance ability.

Stem cells and neuronal stem cells may be isolated from embryonic, post-natal, juvenile or adult neural tissue. Preferably, the neural tissue is mammalian, and more preferably, the neural tissue is human. Isolation, proliferation and passaging of neuronal stem cells from various neural tissues have been described. (Weiss et al., U.S. Pat. Nos. 5,750,376 and 5,851,832; Johe, U.S. Pat. No. 5,753,506;WO 93/01275; WO 94/09119; WO 94/10292; WO 94/16718; Cattaneo et al. (1996) Mol. Brain Res. 42: 161-166; all incorporated herein by reference). In the presence of a proliferation inducing growth factor (i.e., EGF, bFGF, or the like) in suspension culture, neuronal stem cells divide and produce a cluster of undifferentiated cells referred to as a neurosphere. Continued exposure of the neurosphere to the growth factors is an effective method of amplifying a neuronal stem cell. (Weiss et al., U.S. Pat. Nos. 5,750,376 and 5,851,832; Johe, U.S. Pat. No. 5,753,506). Removal of the growth factors results in differentiation into neurons, astrocytes and oligodendrocytes. Neurospheres may be transplanted as neurospheres or dissociated chemically, mechanically (i.e., tituration), and by any other methods that are well known in the art.

Stem cells and neuronal stem cells may be treated with RXR ligands, in culture, as described above. These cells may then be transplanted into mammalian and preferably human subjects in need thereof. Preferably, the subject has a neurodegenerative disease. More preferably, the subject has Alzheimer's Disease, Multiple Sclerosis, Huntington's Disease, Amylotrophic Lateral Sclerosis, or Parkinson's Disease, each of which have been linked to the degeneration of neural cells in particular locations of the central-nervous system.

Stem cells and neuronal stem cells are delivered to the subject by any suitable means known in the art. Methods for the injection of cell suspensions, such as fibroblasts, into the central-nervous system are applicable to the administration of the stem cells. (Bjorklund and Stenevi (eds.) (1987) Neural Grafting in the Mammalian CNS; incorporated herein by reference). As used herein, an “effective amount” of neuronal stem cells for transplantation refers to an amount or number of cells sufficient to obtain the desired effect. The stem cells will generally be administered at concentrations of about 5-50,000 cells/μl. Although injection volumes are preferably about 5-20 μl, transplantation subsequent to surgery may involve volumes that are several fold greater. The number of transplanted cells is only limited by utility and can be determined by a person skilled in the art without undue experimentation.

Transplantation of cells may also utilize known encapsulation technologies, including microencapsulation (U.S. Pat. Nos. 4,352,883; 4,353,888; and 5,084,350; all incorporated herein by reference) and macroencapsulation (U.S. Pat. Nos. 5,284,761, 5,158,881, 4,976,859 and 4,968,733;WO92/19195; WO 95/05452; each incorporated herein by reference). Stem cells may also be transplanted in combination with a capsular device, wherein the device secretes a therapeutically effective molecule or precursor (i.e., pro-drug) that has a growth or trophic effect on the transplanted stem cells, or that induces differentiation of the stem cells towards a particular phenotypic lineage, or a combination thereof. Preferably, the transplanted capsular device contains RXR ligand(s), or a pharmaceutical composition thereof.

The transplanted neuronal stem cells may be isolated from the same species or a species that differs from the recipient. Transplanted cells that are not obtained from the recipient are referred to herein as “heterologous cells”. As used herein, “autologous” refers to cells that are obtained or originate from the recipient. Thus, an autologous donor is the recipient. Also, prior to transplantation, neuronal stem cells may be manipulated in vitro.

Cells used for transplantation are preferably cultured in a completely defined culture medium (serum free) containing the nutrients and hormones necessary to support the cells. As used herein, “completely defined” refers to a medium where all of the components are known. Numerous completely defined culture media are commercially available. A preferred medium comprises a 1:1 mixture of Dulbecco's Modified Eagle's Medium and F12 nutrient (GIBCO) plus 0.6% glucose, 2 mM glutamine, 3 mM sodium bicarbonate, 5 mM HEPES buffer and a defined hormone mix and salt mixture (Sigma; 10% by volume) that includes 25 μg/ml insulin, 100 μg/ml transferrin, 20 μM progesterone, 50 μM putrescine, and 30 nM selenium chloride. The RXR ligand-treated dopamine secreting cells of the present invention can be administered to any animal, preferably human, with abnormal neurological or neurodegenerative symptoms. The symptoms may result from mechanical, chemical, or electrolytic lesions, as a result of experimental aspiration of neural areas, or as a result of aging processes. Particularly preferable lesions in non-human animal models are obtained with 6-hydroxy-dopamine (6-OHDA), 1-methyl-4-phenyl-1,2,3,6 tetrahydropyridine (MPTP), ibotenic acid and the like.

Purified populations of differentiated dopamine-secreting cells that have been treated with RXR ligands, in culture, may be implanted into dopamine-deficient regions of the brain of a recipient. Any suitable method for purifying the cells may be used. Cells may also be implanted with other neural cells. Any suitable method for the implantation of dopaminergic cells or precursor cells near or in the region of dopamine depletion may be used. Methods for the injection of cell suspensions, such as fibroblasts, into the central-nervous system may be employed for the injection of the dopamine-secreting cells prepared by the culture methods disclosed herein. (U.S. Pat. No. 5,082,670 to Gage et al.; Bjorklund and Stenevi (eds.) (1987) Neural Grafting in the Mammalian CNS; both incorporated herein by reference). Xeno and/or allografts may require the application of immunosuppressive techniques or induction of host tolerance to enhance the survival of the implanted cells.

Implantation of neuronal cells may also utilize known encapsulation technologies, including microencapsulation (U.S. Pat. Nos. 4,352,883; 4,353,888; and 5,084,350; all incorporated herein by reference) and macroencapsulation (U.S. Pat. Nos. 5,284,761, 5,158,881, 4,976,859 and 4,968,733; WO92/19195; and WO 95/05452; each incorporated herein by reference). Neuronal cells may also be transplanted in combination with a capsular device, wherein the device secretes a therapeutically effective molecule or precursor that has a growth or trophic effect on the transplanted neuronal cells. Preferably, the transplanted capsular devices contain RXR ligand(s), or a pharmaceutical composition thereof.

4. Identification of Genes Transcriptionally Regulated by RXR in a Neuronal Survival Response Pathway

The present invention also provides a method for identifying a gene that is under the transcriptional control of RXR in dopamine secreting cells, wherein the method comprises the steps of: 1) contacting a neuronal cell with an RXR ligand that activates RXR and an RXR-dependent-response pathway that mediates the increased survival of the dopamine secreting cells; and 2) identifying transcripts that are differentially expressed in the treated cells, wherein the differentially expressed transcripts in the treated cells are encoded by genes under the transcriptional control of RXR. This method contemplates the use of a variety of dopamine secreting cells that are isolated from the central-nervous system, and a variety of RXR ligands. Preferably, the RXR ligands of the present invention specifically activate RXR and RXR-mediated response pathways and do not substantially activate RAR-mediated response pathways. More preferably, the RXR ligand activates the RXR-dependent-response pathway that mediates the increased survival of dopamine secreting cells.

Numerous methods of determining differential gene expression are well known in the art. The present invention contemplates any method of identifying differentially expressed genes. Following RXR ligand-treatment of dopamine secreting cells, RNA is isolated and compared to RNA from untreated cells. Transcripts that are expressed at a relatively higher level in the treated cells are identified. The identification of differentially transcribed genes may be achieved by, i.e., differential display, array analysis or by serial analysis of gene expression (SAGE). Expression patterns of target genes in various tissues and at various stages of development and cell cycle are routinely analyzed by differential display, indexing, subtraction hybridization, or a variety of DNA fingerprinting techniques, and the like. (Vos et al. (1995) Nucleic Acids Research 23: 4407-4414; Hubank et al. (1994) Nucleic Acids Research 22: 5640-5648; Lingo et al. (1992) Science 257: 967-971; McClelland et al., U.S. Pat. No. 5,437,975; Unrau et al. (1994) Gene 145: 163-169; Geng et al. (1998) BioTechniques 25: 434-438; Linskens et al. (1995) Nucleic Acids Research 23: 3244-3251; Lee et al. Proc. Nat. Acad. Sci. U.S.A. 88: 2825-2829; Laing et al. (1992) Science 257: 967-971; Kato (1995) Nucleic Acids Research 12: 3685-3690; each incorporated herein by reference). Microarrays comprising oligonucleotides or polynucleotides for detecting the complementary sequences of expressed genes are also routinely used in the art. (Schena et al. (1995) Science 270: 467-469; DeRisi et al. (1997) Science 278: 680-686; Chee et al. (1996) Science 274: 610-614); each incorporated herein by reference). Recently, the serial analysis of gene expression (SAGE), comprising the excision and concatenation of short sequence tags from cDNAs, followed by conventional sequencing of the concatenated tags, has been successfully used to analyze gene expression and differential gene expression. (Velculescu et al. (1995) Science 270: 484-486; Zhang et al. (1997) Science 276: 1268-1272; Velculescu et al. (1997) Cell 88: 243-251; each incorporated herein by reference).

The present invention also provides a method for identifying a gene that is under the transcriptional control of RXR, wherein the dopamine secreting cells are derived from the retina, olfactory bulb, hypothalamus, dorsal motor nucleus, nucleus tractus solitarious, periaqueductal gray matter, ventral tegmentum, or substantia nigra.

In another embodiment, the RXR ligand is a naturally-occurring ligand. Preferably, the naturally-occurring RXR ligand is 9-cis retinoic acid or docosahexaenoic acid. Other embodiments of the present invention include administration of an RXR ligand that is: 1) isolated from ventral embryonic midbrain cells; 2) contained in conditioned media produced by incubating embryonic ventral midbrain explants in culture media; 3) a naturally-occurring RXR ligand that is endogenous to the dopamine secreting cells to which the RXR ligand is administered; 4) a naturally-occurring RXR ligand that is not endogenous to the neuronal cells to which the RXR ligands is administered; or 5) a non-naturally-occurring-RXR ligand or RXR-ligand analog.

The present invention also provides a method for identifying a gene that is under the transcriptional control of RXR, wherein an RXR ligand or RXR-ligand analog selectively activates RXR-mediated response pathways. In a more preferred embodiment, RXR ligands or RXR-ligand analogs activate RXR and RXR-mediated response pathways, but do not substantially activate RXR-RAR heterodimers and RAR-dependent-response pathways. In the most preferred embodiment, the non-naturally-occurring-RXR ligands or RXR analogs are selected from the group consisting of: LG1069 (Boehm et al. (1994) J. Med. Chem. 37: 2930-2941; Mukherjee et al. (1997) Nature 386: 407-410; both incorporated herein by reference), LG100268 (Boehm et al. (1995) J. Med. Chem. 38: 3146-3155; Mukherjee et al. (1997) Nature 386: 407-410; both incorporated herein by reference), SR11203, SR11217, SR11234, SR11235, SR11236 and SR11237 (Lehmann et al. (1992) Science 258:1944-1946; incorporated herein by reference; see also, FIG. 3).

5. Identification of Agents that Stimulate Ret Expression

In models of Parkinson's disease in both rodents and primates, Ret ligands such as GDNF efficiently influence the survival of dopamine-secreting cells and may likely become important in future therapies for Parkinson's disease. (Björklund et al. (2000) Brain Res. 886: 82-98; Kordower et al. (2000) Science 290: 767-773; Olson (2000) Science 290: 723-724.). Data supports a conclusion that Nurr1 influences responsiveness to these factors by affecting Ret gene expression. For example, relatively small increases in expression of the Ret co-receptor, GFRα1, markedly enhances sensitivity to GDNF in superior cervical ganglion cells. (Heng et al. (2000) J. Neurosci. 20: 2917-2925). Conversely, GFRα1^{+/-} mice are significantly less able to respond to GDNF as shown by the inability of GDNF preparations to counteract the effects of temporary middle-cerebral-artery occlusions in GFRα1 heterozygotes. (Tomac et al. (2000) Neuroscience 95: 1011-1023). Also, Nurr1-heterozygous animals are more sensitive to the effects of 1-methyl-4-phenyl-1,2,3,6-tetrahydropyridine (MPTP), a neurotoxin that selectively induces dopamine-secreting-cell death. (Le et al. (1999) Journal of Neurochemistry 73: 2218-2221). Thus, Nurr1ligands that modulate Nurr1 activity will promote Ret expression and thereby increase survival of dopamine-secreting cells and stimulate dopaminergic-nerve-fiber growth by sensitizing dopamine-secreting cells to endogenous Ret ligands.

The present invention provides methods for screening and identifying an agent that regulates Ret expression and enhances neuronal survival. The method comprises the steps of: 1) contacting a neuronal cell, which coexpresses Nurr1 and Ret, with an agent to be assayed; and 2) measuring Ret expression, wherein a difference in Ret expression compared to a control cell not contacted with said agent indicates the agent regulates Ret expression. In the methods of this invention, an increase in Ret expression indicates that the agent stimulates Ret expression, and a decrease in Ret expression indicates that the agent inhibits Ret expression. This method contemplates the use of a variety of dopamine secreting cells that are isolated from the central-nervous system and coexpress Nurr1 and Ret. Preferably, the cell is a neuronal cell, and more preferably a dopamine-secreting cell. In another embodiment, the cell is Nurr1^{-/-}, expresses Ret, and is transformed or transfected with a vector encoding Nurr1. Preferably, the cell is transformed or transfected with a vector comprising the Nurr1gene and a vector comprising the Ret gene. The transfected or transformed cells, prior to transfection or transformation, may be Nurr1^{-/-} or Ret^{-/-}, or Nurr1^{-/-} and Ret^{-/-}. As used herein, “Nurr1^{-/-} cell” refers to a cell that lacks a functional copy of the Nurr1 gene, and consequently, does not produce Nurr1 protein. As used herein, “Ret−/− cell” refers to a cell that lacks a functional copy of the Ret gene, and consequently, does not produce Ret protein.

Numerous methods of determining gene expression are well known in the art. The present invention contemplates any method of identifying Ret expression. After contacting the agent with the dopamine secreting cells, Ret-specific RNA is measured and compared to Ret-specific RNA from untreated cells. The identification of Ret-specific RNA may be achieved by, i.e., Northern analysis, quantitative PCR, differential display, or array analysis. Expression patterns of target genes in various tissues and at various stages of development and cell cycle are routinely analyzed by differential display, indexing, subtraction hybridization, or a variety of DNA fingerprinting techniques, and the like. (Vos et al. (1995) Nucleic Acids Research 23: 4407-4414; Hubank et al. (1994) Nucleic Acids Research 22: 5640-5648; Lingo et al. (1992) Science 257: 967-971; McClelland et al., U.S. Pat. No. 5,437,975; Unrau et al. (1994) Gene 145: 163-169; Geng et al. (1998) BioTechniques 25: 434-438; Linskens et al. (1995) Nucleic Acids Research 23: 3244-3251; Lee et al. Proc. Nat. Acad. Sci. U.S.A. 88: 2825-2829; Laing et al. (1992) Science 257: 967-971; Kato (1995) Nucleic Acids Research 12: 3685-3690; each incorporated herein by reference). Microarrays comprising oligonucleotides or polynucleotides for detecting the complementary sequences of expressed genes are also routinely used in the art. (Schena et al. (1995) Science 270: 467-469; DeRisi et al. (1997) Science 278: 680-686; Chee et al. (1996) Science 274: 610-614); each incorporated herein by reference).

In another embodiment, Nurr1^{-/-} cells are transformed or transfected with a vector encoding Nurr1 (Law et al. (1992) Mol. Endocrinol. 6: 2129-2135; incorporated herein by reference). The method comprises the steps of: 1) contacting a Nurr1^{-/-} cell, which has been transformed with a vector comprising Nurr1, with an agent; and 2) measuring Ret-gene expression, wherein a difference in Ret expression relative to an untreated cell indicates that the agent regulates Ret expression. This method contemplates any expression vector that enables expression of the encoded Nurr1. Preferably, the cell is transformed or transfected with a vector comprising the Nurr1gene and a vector comprising the Ret gene. The transfected or transformed cells, prior to transfection or transformation, may be Nurr1^{-/-} or Ret^{-/-}, or Nurr1^{-/-} and Ret^{-/-}.

In an other embodiment, cells that do not express either Nurr1 or Ret are transformed or transfected with an expression vector comprising Nurr1 and a vector comprising the Ret gene, including its 5′-regulatory region (Iwamoto et al. (1993) Oncogene 8: 1087-1091; incorporated herein by reference). The method comprises the steps of: 1) contacting a cell with an agent, wherein the cell does not express either Nurr1 or Ret, and wherein the cell is transformed or transfected with a vector comprising the Nurr1 gene and a vector comprising the Ret gene; and 2) measuring Ret expression, wherein a difference in Ret expression compared to a control cell not contacted with said agent indicates the agent regulates Ret expression.

The present invention also provides methods for screening and identifying an agent that regulates Nurr1. The method comprises the steps of: 1) contacting a cell expressing Nurr1 and Ret with an agent to be assayed; and 2) measuring Ret expression, wherein a difference in Ret expression compared to a control cell not contacted with said agent indicates the agent regulates Nurr1. In the methods of this invention, an increase in Ret expression indicates that the agent stimulates Ret expression, and a decrease in Ret expression indicates that the agent inhibits Ret expression. This method contemplates the use of a variety of dopamine secreting cells that are isolated from the central-nervous system and coexpress Nurr1 and Ret. Preferably, the cell is a neuronal cell, and more preferably a dopamine-secreting cell. In another embodiment, the cell is Nurr1^{-/-}, expresses Ret, and is transformed or transfected with a vector encoding Nurr1. Preferably, the cell is transformed or transfected with a vector comprising the Nurr1 gene and a vector comprising the Ret gene. The transfected or transformed cells, prior to transfection or transformation, may be Nurr1^{-/-} or Ret^{-/-}, or Nurr1^{-/-} and Ret^{-/-}.

### 6. Methods of Enhancing Ret Expression-Nurr1 Ligand and RXR Ligand

While RXR is an inactive partner in complex with several nuclear receptors, RXR can be very efficiently activated by its ligand in complex with Nurr1 or NGFI-B, suggesting that one function of these orphan receptors might be to promote ligand-induced signaling by RXR, in vivo. (Perlmann et al. (1995) Genes and Dev. 9:769-782; Forman et al. (1995) Cell 81: 541-550; Chen et al. (1996) Nature 382: 819-822; Kurokawa et al. (1994) Nature 371: 528-531; Minucci et al. (1997) Mol. Cell. Biol. 17(2): 644-655; Vivat et al. (1997) EMBO J. 16(18): 5697-5709; Botling et al. (1997) J. Biol. Chem. 272 (14): 9443-9449). Nurr1 forms heterodimers with RXR and binds to certain retinoic-acid-responsive elements. (Perlmann et al. (1995) Genes and Dev. 9:769-782; Forman et al. (1995) Cell 81: 541-550).

The present invention provides methods of increasing the expression of Ret in dopamine secreting cells, comprising the steps of administering an agent to a subject in need thereof, wherein the agent increases Ret expression. Preferably, the agent is a Nurr1 ligand or an RXR ligand. The preferred RXR ligand is a naturally-occurring RXR ligand, such as 9-cis retinoic acid and docosahexaenoic acid or RXR ligands that are isolated from ventral embryonic midbrain cells, or contained in conditioned media produced by incubating embryonic ventral midbrain explants in culture media. Other preferred RXR ligands include: i.e., naturally-occurring RXR ligands endogenous to the neuronal cells to which the RXR ligands are administered; naturally-occurring RXR ligands that are not endogenous to the dopamine-secreting cells to which the RXR ligand is administered; and a non-naturally-occurring-RXR ligand or RXR-ligand analog. A more preferred RXR ligand is selected from the class of RXR ligands and RXR-ligand analogs that selectively activate RXR-mediated response pathways. These RXR ligands include naturally-occurring analogs that are either synthesized or isolated from a biological sample, or non-naturally-occurring-RXR ligands and RXR-ligand analogs, such as LG1069 (Starrett, Jr. et al. U.S. Pat. No. 5,559,248; Boehm et al. (1994) J. Med. Chem. 37: 2930-2941; Mukherjee et al. (1997) Nature 386: 407-410; all incorporated herein by reference), LG100268 (Starrett, Jr. et al. U.S. Pat. No. 5,559,248; Boehm et al. (1995) J. Med. Chem. 38: 3146-3155; Mukherjee et al. (1997) Nature 386: 407-410; all incorporated herein by reference), SR11203, SR11217, SR11234, SR11235, SR11236 and SR11237 (Starrett, Jr. et al. U.S. Pat. No. 5,559,248; Lehmann et al. (1992) Science 258:1944-1946; both incorporated herein by reference; see also, FIG. 3).

The present invention provides methods of increasing the expression of Ret in dopamine secreting cells, comprising the steps of administering an agent to a subject in need thereof, wherein the agent activates Nurr1, thereby increasing the expression of Ret in dopamine secreting cells. Preferably, the agent is a Nurr1 ligand or an RXR ligand. The preferred RXR ligand is a naturally-occurring RXR ligand, such as 9-cis retinoic acid and docosahexaenoic acid or RXR ligands that are isolated from ventral embryonic midbrain cells, or contained in conditioned media produced by incubating embryonic ventral midbrain explants in culture media. Other preferred RXR ligands include: i.e., naturally-occurring RXR ligands endogenous to the neuronal cells to which the RXR ligands are administered; naturally-occurring RXR ligands that are not endogenous to the dopamine-secreting cells to which the RXR ligand is administered; and a non-naturally-occurring-RXR ligand or RXR-ligand analog. A more preferred RXR ligand is selected from the class of RXR ligands and RXR-ligand analogs that selectively activate RXR-mediated response pathways. These RXR ligands include naturally-occurring analogs that are either synthesized or isolated from a biological sample, or non-naturally-occurring-RXR ligands and RXR-ligand analogs, such as LG1069 (Starrett, Jr. et al. U.S. Pat. No. 5,559,248; Boehm et al (1994) J. Med. Chem. 37: 2930-2941; Mukherjee et al. (1997) Nature 386: 407-410; all incorporated herein by reference), LG100268 (Starrett, Jr. et al. U.S. Pat. No. 5,559,248; Boehm et al. (1995) J. Med. Chem. 38: 3146-3155; Mukherjee et al. (1997) Nature 386: 407-410; all incorporated herein by reference), SR11203, SR11217, SR11234, SR11235, SR11236 and SR11237 (Starrett, Jr. et al. U.S. Pat. No. 5,559,248; Lehmann et al. (1992) Science 258:1944-1946; both incorporated herein by reference; see also, FIG. 3).

### EXAMPLES

### Example 1

### Tissue Explants

Brain tissue explants were produced by isolating embryos from pregnant NMRI mice that were sacrificed by cervical dislocation. Whole midbrains were dissected and the explants were placed directly on transfected JEG3 cells or finely dissected into separate ventral and dorsal pieces and placed onto the cells. (Perlmann et al (1995) Genes and Dev. 9, 769-782; Mata de Urquiza et al. (1999) Proc. Natl. Acad. Sci USA 96: 13270-13275; Zetterström et al. (1996) Mol. Endo. 10: 1656-1666).

### Example 2

### Conditioned Media

Tissue conditioned media was produced by incubating finely dissected embryonic cortex and midbrain tissue, overnight in tissue culture media (serum free minimal essential media, MEM). Following incubation, tissue was removed from the media by centrifugation. This conditioned media was then added to cultured cells which are transfected with RXR-, nuclear receptor- (RXR- heterodimer partner), and reporter constructs.

### Example 3

### Transfection

Expression vectors that encode the ligand binding domain of receptor Nurr1, an orphan- nuclear receptor that heterodimerizes with RXR, were used in these experiments. RXR is a permissive heterodimerization partner in RXR-Nurr1heterodimers, and is efficiently activated by ligand when in such complexes.

Human choriocarcinoma cells, JEG3, were transfected in triplicate in 24-well plates. (Perlmann et al. (1995) Genes and Dev. 9: 769-782; incorporated herein by reference). Each well was transfected with 100 ng of the gal4-binding luciferase reporter construct, MH100-tk-luc, 100 ng of CMX-RXR (Perlmann et al. (1995) Genes and Dev. 9: 769-782), and 100 ng of either CMX-gal4-Nurr1 (Perlmann et al. (1995) Genes and Dev. 9: 769-782), or CMX-gal4-Nurr1 dim⁻. Site directed mutagenesis was used to generate a full-length cDNA encoding Nurr1 dim⁻, wherein the proline at amino acid position 560 was changed to alanine. Proline 560 is within the following segment of Nurr1 sequence: ⁵⁵³SKLLGKLPELR⁵⁶³ (SEQ ID NO: 1). Nurr1 dim is unable to heterodimerize with RXR.

In reporter assays of conditioned media and 9-cis retinoic acid titration experiments, a CMX-gal4-RAR construct (Perlmann et al. (1995) Genes and Dev. 9: 769-782) was also used. In all experiments, CMX-βgal (200 ng) (Perlmann et al. (1995) Genes and Dev. 9, 769-782) was used as an internal control. Following transfection of JEG3 cells (6 hours post-transfection), calcium precipitate was removed by washing with fresh medium. Tissue explants or conditioned media were then added to the cells. After 24 hours, cells were harvested and assayed for luciferase and reference β-galactosidase activity on a luminometer/photometer.

### Example 4

### Primary Ventral Midbrain Cultures

Ventral midbrains from rat embryos at stage E15.5 were dissected, mechanically dissociated, and plated on poly-D-lysine coated 12 well plates in serum-free medium (N2; consisting of a 1:1 mixture of F12 and MEM with 5 μg/ml insulin, 100 μg/ml transferrin, 60 μM putrescine, 20 nM progesterone, 30 nM selenium, 6 mg/ml glucose and 1 mg/ml bovine serum albumin).

Ventral midbrain, cortex and hippocampus from rat embryos at stage E14.5-15.5 were dissected, mechanically dissociated and plated on poly-D-lysine coated 12 or 24 well plates in serum free media (N2, consisting of a 1:1 mixture of MEM (+15 mM HEPES buffer) and Ham's F12 medium (+6 mg/ml glucose, 1 mg/ml bovine serum albumin, 5 ug/ml insulin, 100 ug/ml transferrin, 60 μM putrescine, 20 nM progesterone, 30 nM selenium and 1 mM glutamine). Ligands (all-trans RA, 9-cis retinoic acid, LG268, docosahexanoic acid (DHA), SR11237, TTNPB, and LB1208; stock solutions in DMSO) were diluted to working concentrations in N2, added to cells and incubated 3-5 days. GDNF was dissolved in phosphate-buffered saline (PBS) containing 1 mg/ml bovine serum albumin. These experiments were conducted in triplicate. Paraformaldehyde fixed cultures were then incubated over night with anti-tyrosine hydroxylase (TH) (1:1000, Pel-Freez), anti-NeuN (1:200, Chemicon) or anti-Nurr1 (1: 10,000, Santa Cruz) antibodies in PBS containing 0.5% (TH and NeuN) or 10% (Nurr1) fetal calf serum and 0.3% triton X100. Following rinses, cultures were incubated with biotinylated secondary antibody followed by detection of immuno-staining using the ABC immunoperoxidase kit from Vector. Cells were counted using brightfield microscopy (Nikon Eclipse EM 300). Cell counting was performed independently by two individuals to derive unbiased results.

Bromodeoxyuridine (BrdU) was added in relevant experiments 4-6 hours before fixation. After fixation with 4% paraformaldehyde for 45 minutes, cells were rinsed in PBS. BrdU incorporation was detected by incubating cells with 2 M hydrochloric acid prior to digesting with 0.25% pepsin (Sigma). Following PBS rinses, cells were incubated in blocking solution (0.5% bovine serum albumin, 0.5% Tween-20 in PBS), and thereafter incubated with anti-BrdU antibody (Dako A/S; diluted 1:20 in blocking solution) at 4° C. overnight. Antibody-binding was detected with anti-mouse CY3-conjugated IgG (Jackson ImmunoResearch Labs Inc.). Cells were analyzed using inverted epifluorescent microscopy.

### Example 5

### Identification of RXR Ligand in Ventral Embryonic Midbrain

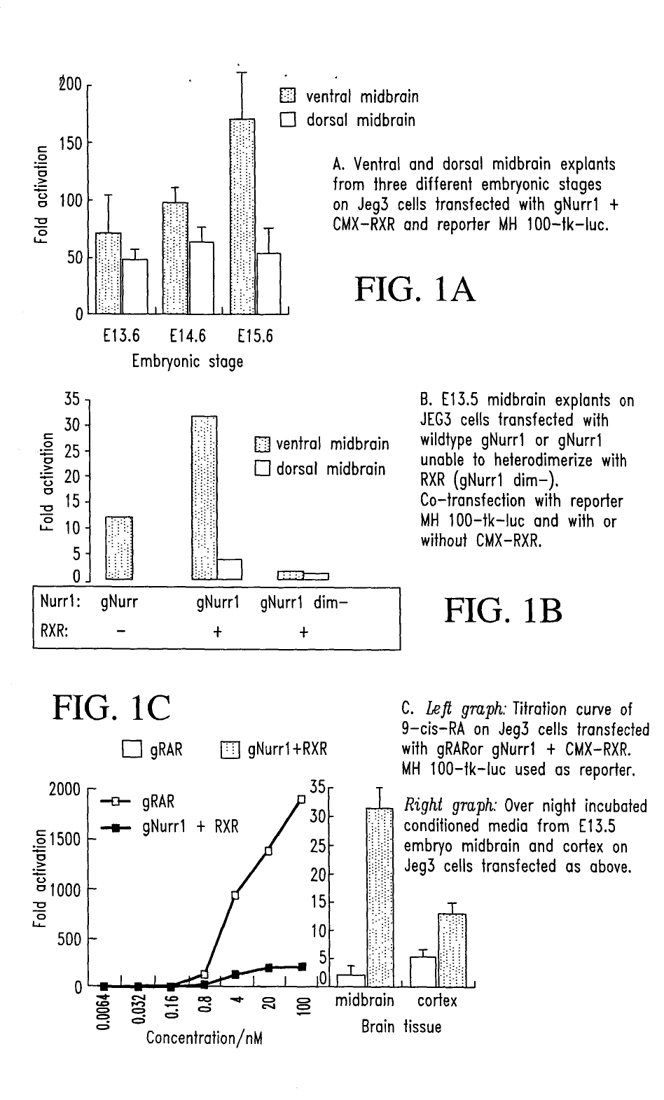

Reporter experiments were performed as previously described. (Perlmann et al. (1995) Genes and Dev. 9, 769-782). Briefly, an expression vector encoding a derivative of the orphan-nuclear receptor Nurr1 (GAL4-Nurr1), was cotransfected into JEG3 cells with an expression vector encoding RXR and a reporter expression vector containing GAL4 binding sites in operable linkage with a luciferase reporter gene. GAL4-Nurr1 and RXR dimerize in transfected cells and efficiently promote transcription from the reporter gene upon binding of RXR ligand. (Mata de Urquiza et al. (2000) Science 290: 2140-2144; Perlmann et al. (1995) Genes and Dev. 9: 769-782; both incorporated herein by reference). Explants were produced from brains at different embryonic stages, as discussed above. The explants were cultured with the transfected cells. After coculturing, cells were harvested, lysed and assayed for luciferase reporter gene activity. FIG. 1A shows that the luciferase gene is induced by an RXR ligand in explants from embryonic mouse midbrain. The activity is higher in cultures containing ventral explants, which corresponds to a region rich in dopamine-secreting cells.

Significantly less activation was observed (FIG. 1B), in the absence of RXR. This demonstrates that activation is dependent on RXR heterodimerization with GAL4-Nurr1. In addition, Nurr1 dim⁻ failed to activate the reporter (FIG. 1B). These results identify the existence of an RXR ligand in embryonic ventral midbrain cells.

Although 9-cis retinoic acid is an efficient ligand for both RAR and RXR, the ventral midbrain activity is unable to activate RAR (FIG. 1C). Thus, the RXR ligand from embryonic ventral midbrain is distinct from 9-cis retinoic acid.

### Example 6

### RXR Ligand SR11237 Increases Neuronal Cell Survival in vitro

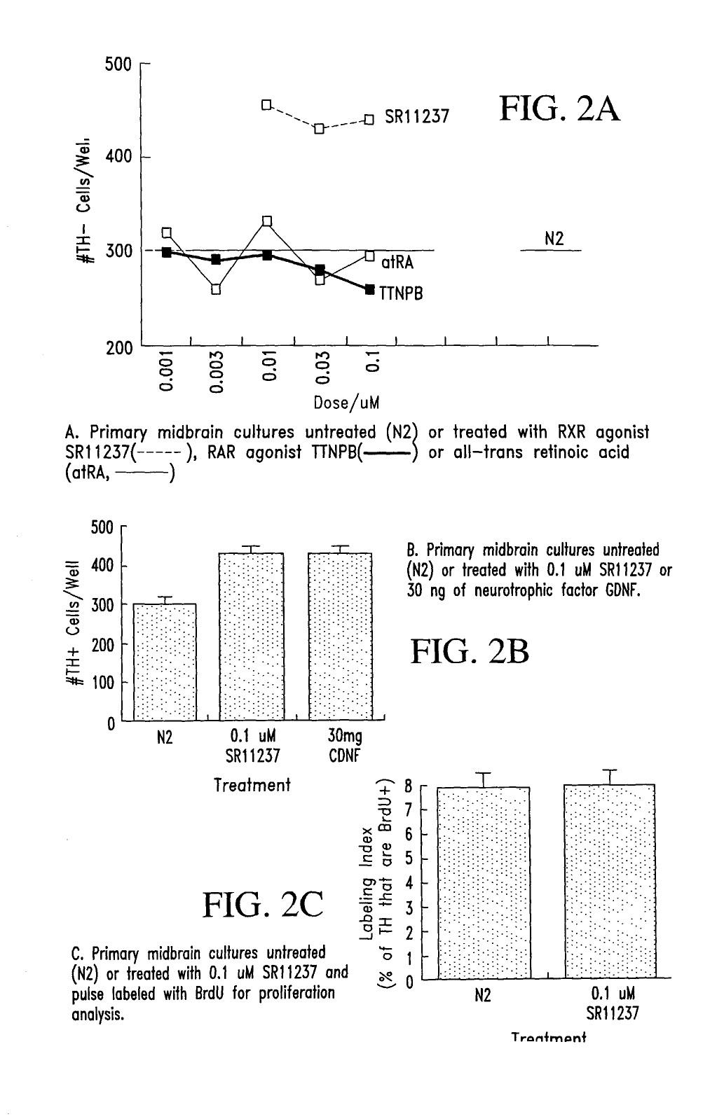

Primary ventral midbrain cultures were established to analyze the effects of RXR ligands on dopamine secreting cells in culture. Interestingly, treating the cultures with a synthetic RXR ligand SR11237 (Lehmann et al. (1992) Science 258:1944-1946; incorporated herein by reference; see also FIG. 3) resulted in increased dopamine secreting cells after a 72 hour incubation, as compared to incubation with an RAR ligand (i.e., TTNPB), all-trans retinoic acid, or in the absence of an RXR ligand. (FIG. 2A). At an SR11237 concentration of 0.1 μM, the increase in neuronal cell number is similar to the effects achieved by GDNF treatment (FIG. 2B). The increased number of cells is not due to increased proliferation induced by SR11237 (FIG. 2C). In conclusion, the synthetic RXR ligand SR11237 promotes dopamine-cell survival in primary-cell cultures, in vitro.

### Example 7

### Nurr1-Mutant Mice

The generation of Nurr1 mutant mice was described Wallën et al. (Wallën et al. (1999) Exp. Cell Res. 253: 737-746.). Nurr1 heterozygous females were bred with Nkx6.2-tau-lacZ males for generation of mice heterozygous for the two mutations and these were in turn crossed with Nurr1 heterozygous mice for generation of Nkx6.2-tau-lacZ^{+/-} Nurr1^{+/+} and Nkx6.2-tau-lacZ^{+/-} Nurr1^{+/+} embryos. Mice were mated and females checked for vaginal plugs. Pregnant females were sacrificed by cervical dislocation and embryos collected from desired stages. For in situ hybridization, embryos were rapidly fresh frozen. For immunohistochemical analyses, embryos were fixed 1-24 hours in 4% phosphate-buffered paraformaldehyde and stored in 30% sucrose. Genotyping was on tail- and amnion-derived DNA by PCR. Littermates were used in all comparative experiments. Cryosections on ProbeOn (Fisher Scientific) and SuperFrost Plus (Menzel-Gläzer) slides were prepared at 14-25 μm thickness.

### Example 8

### Nurr1-Specific Antibody

A GST-fusion protein of the Nurr1-ligand-binding domain was prepared according to manufacturer's recommendations (Pharmacia). Rabbits were given a subcutaneous injection of 150 μg fusion protein in Freund's complete adjuvant (Gibco Life Technologies) followed by booster injections every 2 weeks. For isolation of Nurr1-specific IgG, the Nurr1-GST fusion protein was coupled to CNBr-activated Sepharose and used for affinity purification of immunoglobulin from the immune sera. Nurr1 -like immunoreactivity was not detected in Nurr1 mutant embryos.

### Example 9

### X-Gal Staining and Treatment of Embryos

Following dissection, embryos were fixed in 0.2% glutaraldehyde in phosphate-buffered saline and incubated at 37° C. overnight in a staining solution (2 mM MgCl₂, 0.002% NP-40, 0.01% sodium deoxycholate, 5 mM K4Fe(CN)₆, 5 mM K₃Fe(CN)₆) with 1 mg/ml X-gal (5-bromo-4-chloro-3-indoyl-β-D-galactopyranoside). Embryos were post-fixed in 4% PFA and placed in sucrose. Skin was removed to expose cranial nerve staining.

### Example 10

### Microscopic Evaluation and Image Collection

Analyses were performed on multiple embryos and pups and observations were confirmed by at least two persons. Structures were identified using published anatomical atlases (Altman et al. (1995) Atlas of prenatal rat brain development. CRC Press, Boca Raton; Kaufman (1992) The atlas of mouse development. Academic Press, San Diego; Paxinos et al. (1994) Atlas of the developing rat nervous system. Academic Press, San Diego; Paxinos et al. (1986) The Rat Brain in Stereotaxic Coordinates. Academic Press, Australia). For section analyses, data were evaluated and images were collected using confocal microscope (Axiovert 100M, Zeiss) or regular microscopy (Eclipse E1000M, Nikon) coupled to a digital camera (Spot2, Diagnostic Instruments Inc.). For whole-mount embryos, a dissection microscope (Nikon SMZ-2B) was used with digital camera.

### Example 11

### In Situ Hybridization

Slides were incubated with end-labeled oligonucleotide probes at 42° C. for 16-18 hours, rinsed and dipped in photo-emulsion (NTB2, Kodak) as previously described (Dagerlind et al. (1992) Histochemistry 98: 39-49; Zetterström et al. (1996) Endocrinol. 10: 1656-1666; Zetterström et al. (1996) Mol. Brain Res. 41: 111-120). Exposure was for 6 weeks followed by cresyl-violet staining and mounting. Oligonucleotide probes to the following sequences were used: Nurr1 (both wild-type and mutant) 1430-1477 (Law et al. (1992) Mol. Endocrinol. 6: 2129-2135; Wallën et al. (1999) Exp. Cell Res. 253: 737-746), Phox2a 29-78 (Valarche et al. (1993) Development 119: 881-896), ChAT 1818-1853 (Brice et al. (1989) J. Neurosci. Res. 23: 266-273), Ret 2527-2576 (Iwamoto et al. (1993) Oncogene 8: 1087-1091), GFRα1 805-851 (Jing et al. (1996) Cell 85: 1113-1124), TH 1441-1478 (Grima et al. (1985) Proc. Natl. Acad. Sci. USA 82: 617-621).

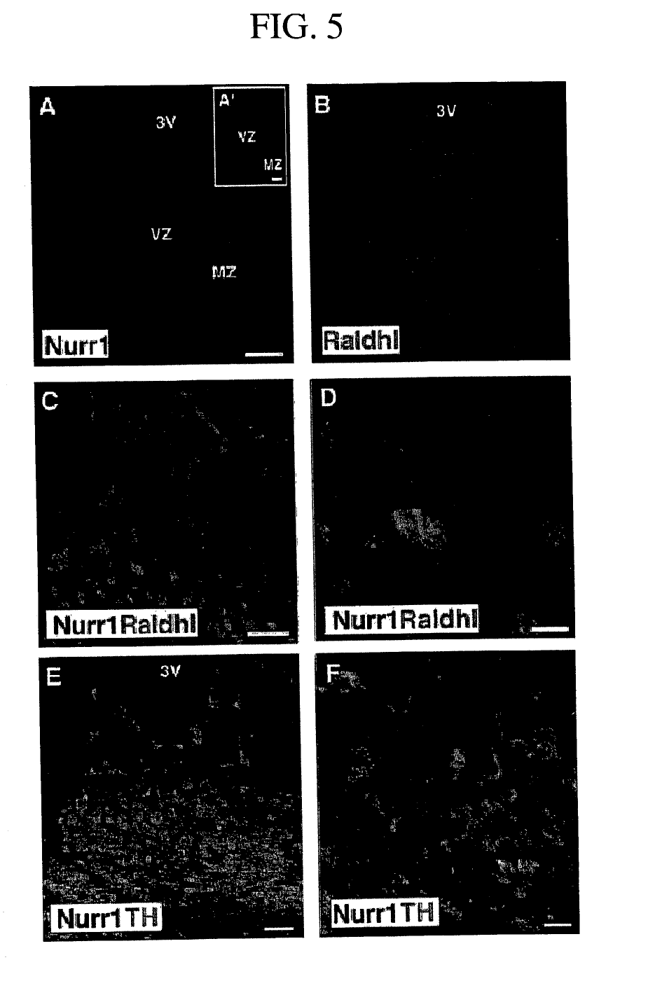

Nurr1 mRNA expression in the mouse has previously been analyzed by in situ hybridization. Using a rabbit polyclonal antibody raised against the Nurr1 carboxy-terminal domain, Nurr1 immunoreactivity was detected in the E12.5 mouse embryonal VMB. Distinct nuclear labeling outside the ventricular zone correlates well with the distribution of Nurr1 mRNA (FIG. 5A). Aldehyde dehydrogenase I (RaldhI, also known as AHD2; Lindahl et al.(1984) J. Biol. Chem. 259: 11991-11996) has been suggested as a marker for proliferating dopaminergic progenitors (Wallén et al. (1999) Exp. Cell Res. 253: 737-746) and is expressed in both the proliferating cells in the ventricular zone and the mantle layer (FIG. 5B). Codetection of Nurr1 and RaldhI shows strong cytoplasmic RaldhI immunoreactivity in the proliferative ventricular zone as well as in the mantle layer of differentiating Nurr1 expressing cells. Nurr1 and RaldhI immunoreactivity are perfectly colocalized in cells outside of the ventricular zone, firmly establishing RaldhI as a marker for dopaminergic progenitors (FIGS. 5C and D). Colocalization of nuclear Nurr1 and cytoplasmic tyrosine hydroxylase (TH) immunoreactivity is identified in cells that have migrated further from the ventricular zone (FIGS. 5E and F). No Nurr1 immunoreactivity was detected in sections of Nurr1mutant embryos, demonstrating the specificity of the antibody.

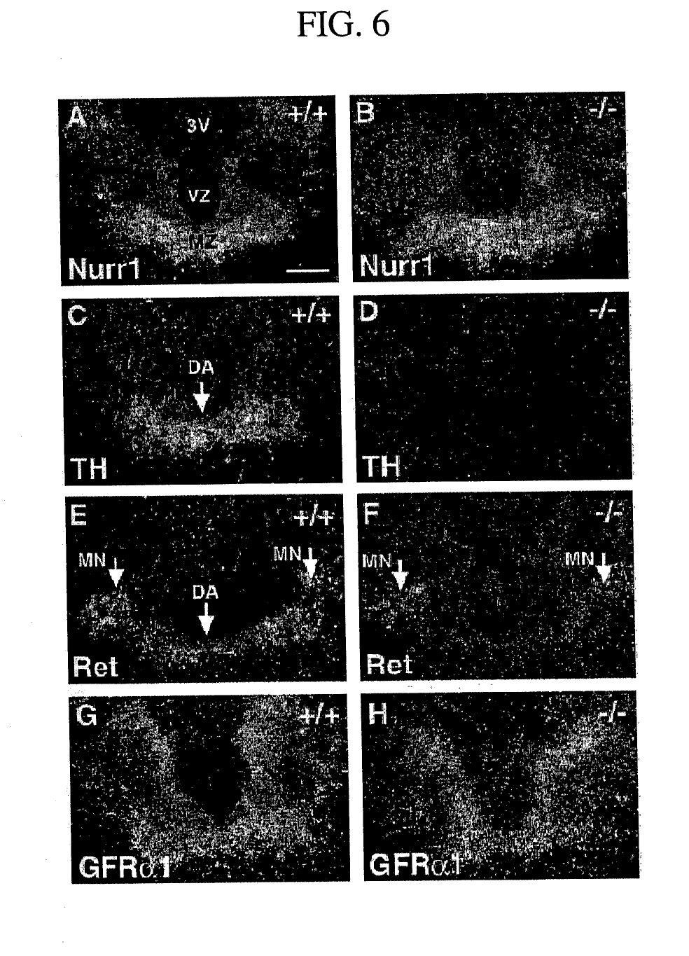

Expression of Ret and GFRα1, the Ret coreceptor, were analyzed to identify Nurr1 -regulated genes that contribute to the Nurr1-deficient dopaminergic-cell phenotype. Ret mRNA expression can be detected in VMB by in situ hybridization in a pattern that is similar to that of TH mRNA at E11.5 (FIG. 6). At this stage, Nurr1 mRNA is expressed in a similar domain in the medial VMB and, additionally, in less mature cells closer to the ventricular cell layer (FIG. 6A). In the Nurr1 mutant, medial VMB cells can be detected with a probe that recognizes a transcript originating from the disrupted Nurr1 locus (FIG. 6B; Wallën et al. (1999) Exp. Cell Res. 253: 737-746). TH and Ret are both induced at around E11.5, one day after the appearance of Nurr1 (FIGS. 6C and E). Already at this early stage of development, Nurr1 deficiency results in the loss of Ret mRNA expression (FIG. 6F). Notably, Ret can still be detected in developing midbrain motor neurons (FIG. 6F) that are normally located laterally to the dopamine-secreting cells and are intact in the Nurr1 -mutant midbrain. (Wallën et al. (1999) Exp. Cell Res. 253: 737-746). Analyses of VMB by in situ hybridization at several different developmental stages—E13.5, E16.5, and newborn-reveal that Ret is not expressed in developing dopamine-secreting cells in Nurr1 -mutant mice. This finding was also confirmed by reverse-transcriptase-coupled polymerase chain reaction of dissected VMB from wild-type and Nurr1-deficient embryos, respectively. Thus, in addition to TH (FIG. 6D), Ret is a second gene that is deregulated from earliest stages in the Nurr1-mutant medial VMB, where dopamine-secreting cells normally develop. In contrast, GFRα1 is induced even in the absence of Nurr1 (FIGS. 6G and H), although this expression, similar to other dopaminergic markers, is abolished at later stages (E16.5) of development in mutant animals. In conclusion, impaired Ret mRNA expression in VMB dopaminergic neurons represents an early deficiency that is characteristic of Nurr1^{-/-} embryos.

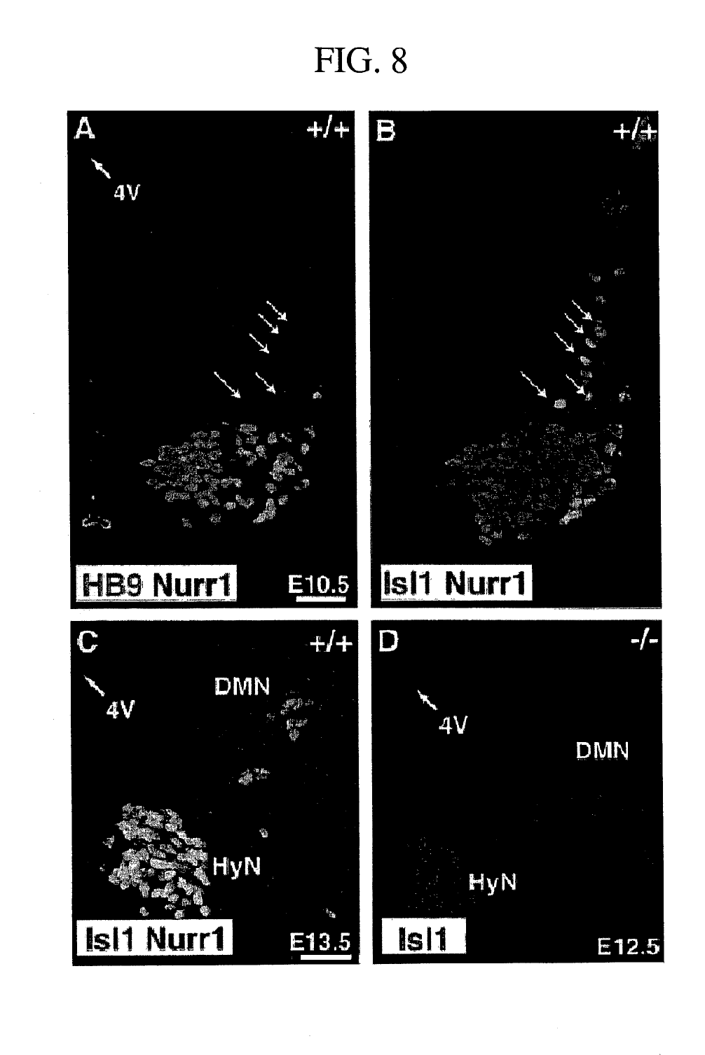

Nurr1 expression in the brainstem was analyzed during development. Colocalization of Nurr1 and markers for somatic-hypoglossal neurons (HB9 and Islet 1) and visceral motor neurons (Islet 1) (Briscoe et al. (2000) Cell 101: 435-445; Ericson et al.(1997) Cell 90; Tsuchida et al.(1994) Cell 79: 957-970)—at E10.5—demonstrated that, although Nurr1 is not expressed in hypoglossal cells (FIG. 8A), it is expressed at this developmental stage in dorsally-migrating Islet 1-expressing visceral motor neurons (FIG. 8B). Nurr1 expression continues at El 3.5 as the visceral motor neurons are detected at a more lateral site close to the fourth ventricle (FIG. 8C). At this stage, Islet1 is almost completely down-regulated in the DMN (FIG. 8C). Some weakly Nurr1 -positive cells can also be detected in the area of the hypoglossal nucleus. In the Nurr1 -mutant hindbrain, DMN-Islet-1-positive cells are present (E12.5) and are localized laterally of the fourth ventricle at a normal location (FIG. 8D). Nurr1 is thus an early molecular marker for DMN cells, but Nurr1 deficiency does not lead to an apparent abnormal cellularity of this nucleus.

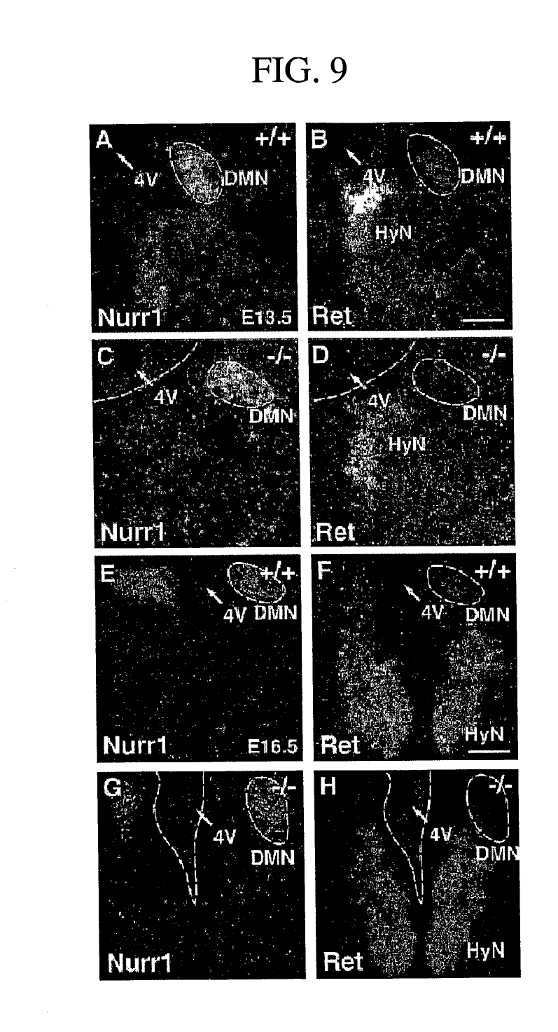

In the wild-type embryo, Nurr1 and Ret mRNA expression can be detected at E13.5 in the DMN (FIGS. 9A and B). Generally, Ret expression is lower compared to the Nurr1 expression in this nucleus. Although the disrupted Nurr1 transcript generated in the Nurr1 mutants can be detected (FIG. 9C), demonstrating the presence of the DMN cells, Ret expression is below detectable levels in the Nurr1 -mutant DMN (FIG. 9D). Similarly, at E16.5, Ret expression is detected in the wild-type DMN, but is absent in Nurr1-mutant embryos (FIGS. 9E-H). Ret expression is normal in the ventral horns of the spinal cord and ureteric buds of the kidney, sites where Ret and Nurr1 are not coexpressed. In conclusion, both embryonic and sustained Ret expression is dependent on Nurr1 from early developmental to postnatal stages in both regions where these two genes are colocalized. Thus, Nurr1 appears to influence Ret-dependent development as well as well as postnatal Ret-dependent functions in dopamine-secreting cells.

### Example 12

### Immunohistochemistry

Slides were air-dried, washed in phosphate-buffered saline, and incubated with blocking solution (3% bovine serum albumin or 0.1% fetal calf serum, 0.3% Triton-X100 in phosphate buffered saline or 10 mM HEPES buffer) before overnight incubation with primary antibody (AHD 2/RaldhI 1:400, beta-galactosidase 1:1000, HB9 1:100, Islet 11:1000, Nurr1 1:2000, pgp9.5 1:400 (Biogenesis), Ret 1:50 (Immuno-biological laboratories Co. Ltd), TH 1:100 and VAChT 1:1000 (Chemic on Int. Inc); each diluted in blocking solution at 4° C.). Following rinsing, the slides were incubated with secondary antibody at 1:200 dilution (Alexa Fluor 488 and 594 IgG (Molecular Probes) or CY3-conjugated IgG, (Jackson ImmunoResearch Laboratories)) for 1 hour. After rinsing, slides were mounted with Vectashield mounting medium (Vector).

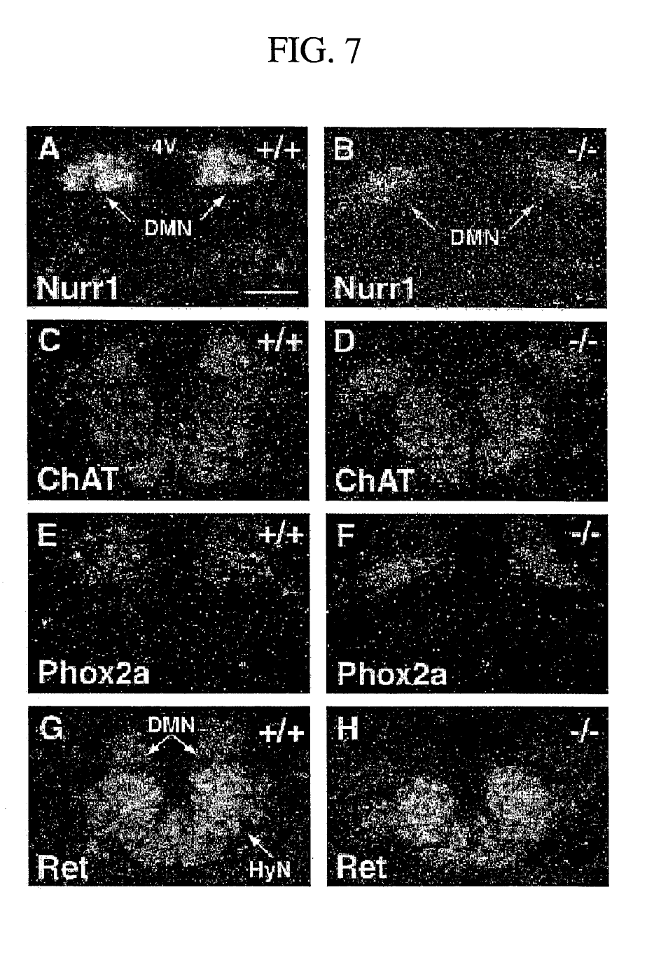

Although DMN cells are generated in Nurr1 -null mice, we examined the possibility that the loss of Nurr1 and the deregulation of Ret, and possibly other genes, produces a functional deficiency of the vagus nerve or inability to correctly innervate peripheral targets. Immunohistochemical analysis localized strong Nurr1 expression to the DMN at E12.5 (FIG. 8). Weaker expression could be detected in the nucleus ambiguus, which is an additional cranial nucleus that contributes efferents to the vagus nerve. In newborn mice, Nurr1 expression was only detected in DMN (FIG. 7).

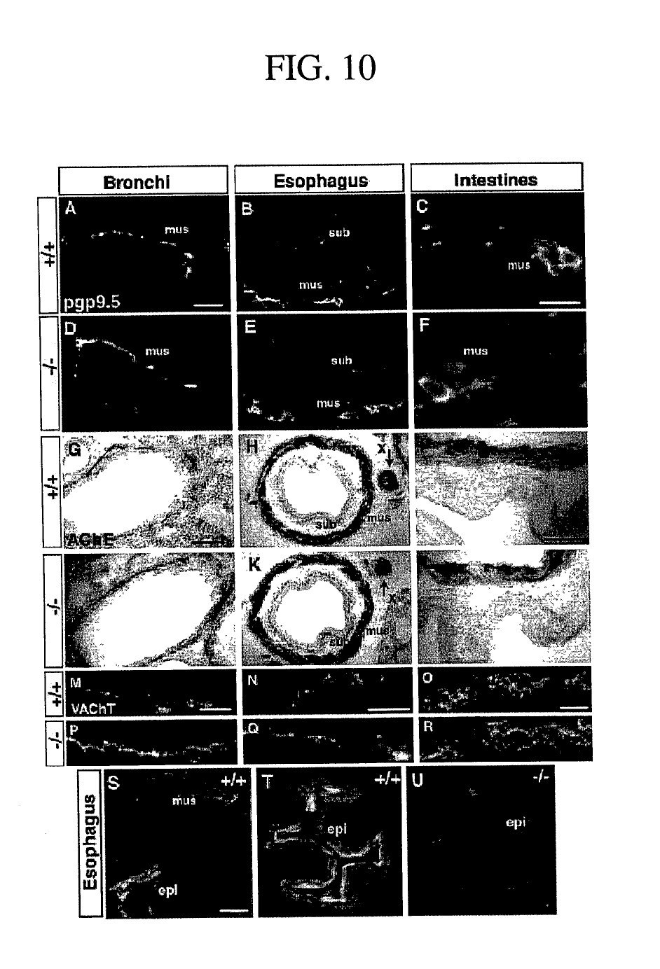

Although the vagus nerve could be visualized by immunolabeling for a general neuronal marker—pgp9.5—in both wild-type and Nurr1^{-/-} embryos at E15.5 (FIGS. 10A and B), it was important to specifically analyze DMN-efferent fibers, which correspond to about 20% of total vagus nerve fibers. For this reason, mice heterozygous for an insertion of a tau-lacZ reporter gene in the locus of the Nkx6.2 gene were interbred with Nurr1 heterozygous mice. Nkx6.2 encodes a homeodomain transcription factor expressed in nucleus ambiguus and DMN, but not in other neurons of the vagus nerve (Qiu et al. (1998) Mech. Dev. 72: 77-88). As expected, a subset of pgp9.5 positive fibers (FIG. 10A) in horizontal cross-sections at E15.5 show immunoreactivity for lacZ-encoded β-galactosidase protein in Nkx6.2^{+/-}Nurr1^{+/+}nerve bundles (FIG. 10C). The pattern is virtually identical in Nkx6.2^{+/-} Nurr1^{+/+} nerves, indicating no gross-outgrowth deficiency of vagus nerve efferents (FIG. 10D). Whole mount x-gal staining at E12.5, however, revealed a subtle abnormality characterized by disorganized fiber tracts at the cranial nuclei exit points (FIGS. 10E-H) in Nkx6.2^{+/-}Nurr1^{-/-} embryos. Although the same number of fibers are present in both genotypes, they appear extended in length in the Nkx6.2^{+/-}Nurr1^{-/-} mice compared with the Nkx6.2^{+/-}Nurr1^{+/+} mice. Thus, although slightly distorted, no severe abnormality in the formation of vagus nerve efferent fibers is apparent in Nurr1 null mutant mice.

### Example 13

### Acetylcholine Esterase Activity

Sections were immersed in ice-cold 4% PFA for 15 minutes, washed in phosphate-buffered saline and incubated in staining solution (38 mM sodium acetate, 0.012% acetic acid, 4.8 mM sodium citrate, 3 mM copper sulphate, 0.08 mM tetra isopropyl pyrophosphoramide (Sigma), 0.5 mM potassium ferricyanide, 0.87 mM acetylthiocholine iodide) for 3 hours after which they were rinsed in water, dehydrated and mounted.

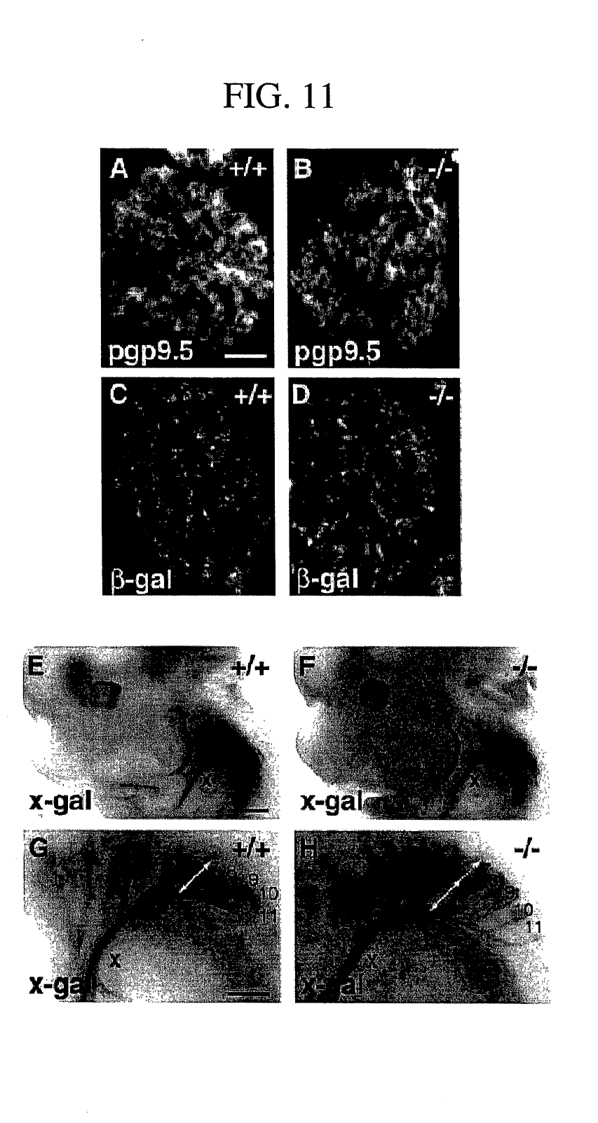

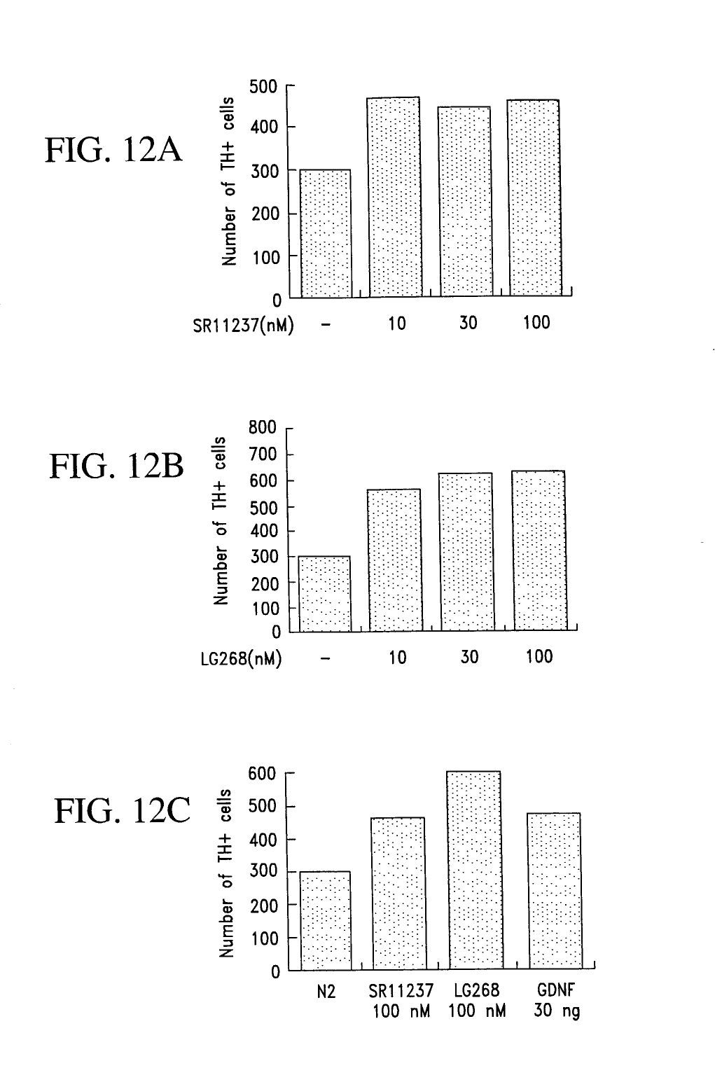

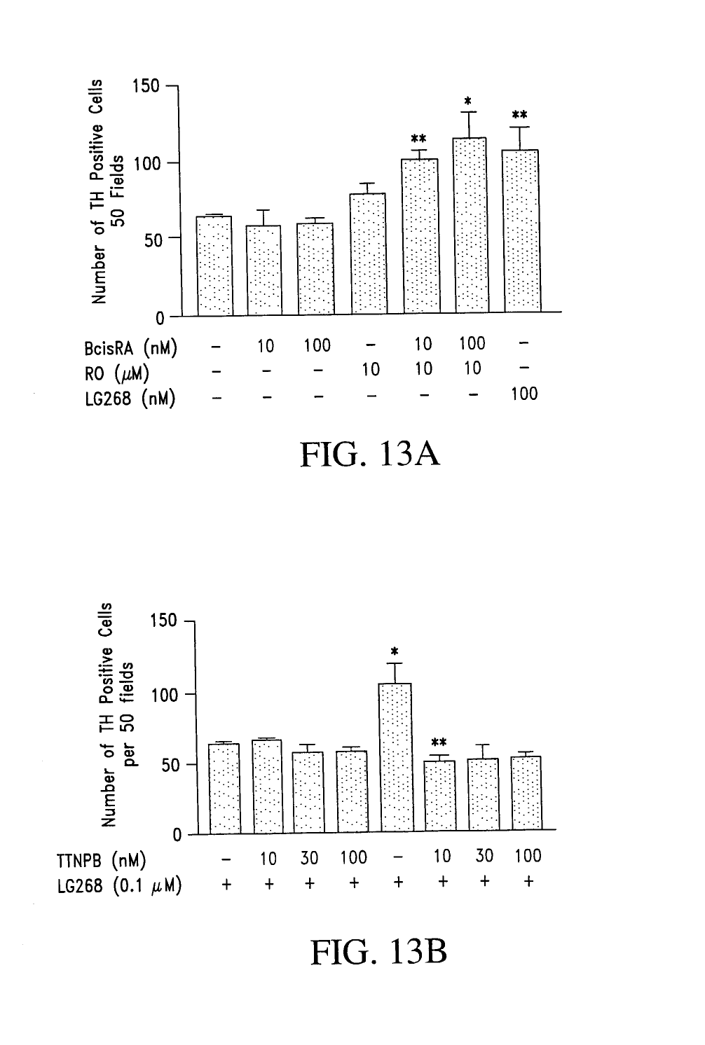

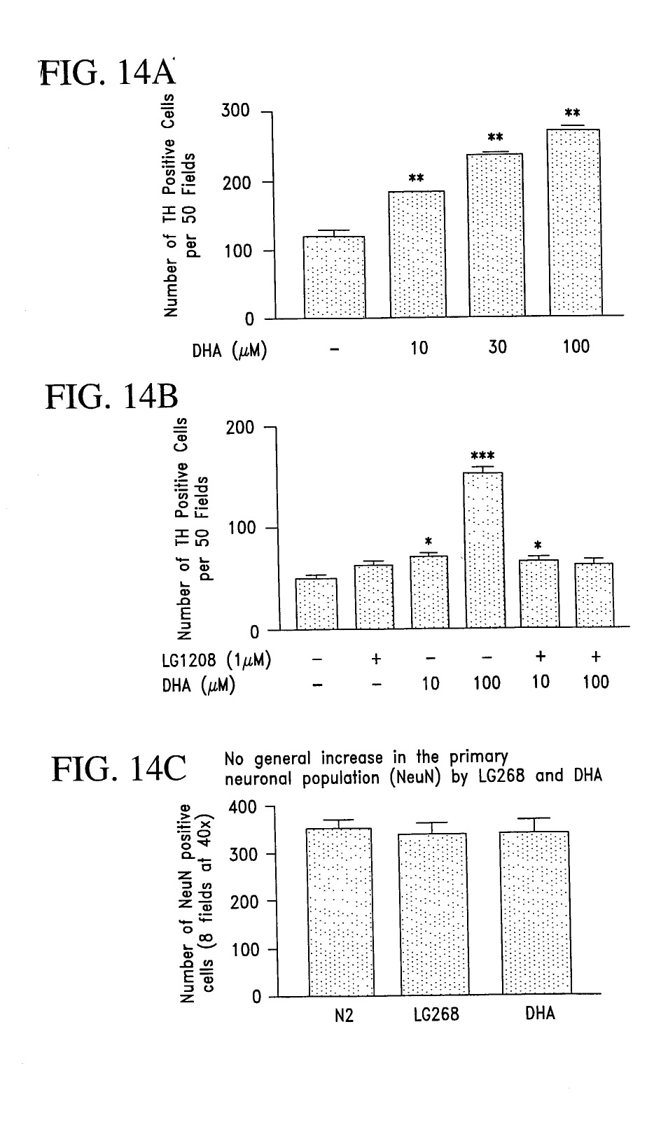

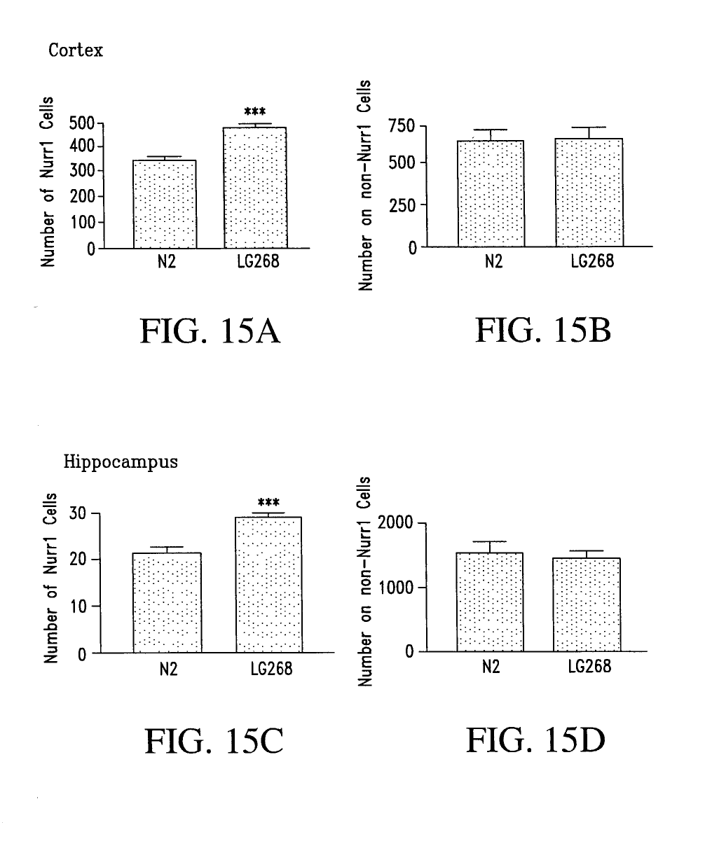

Ganglia of the lungs, heart and gastrointestinal tract are primary target areas of preganglionic-vagus-nerve fibers. Immunohistochemistry was used to analyze innervation from the vagus nerve by detection of pgp9.5, a general neuronal marker, and cholinergic innervation was determined by acetylcholine esterase (ACHE) activity and immunoreactivity to vesicular-acetylcholine transferase (VAChT) located in nerve terminals. Screening of target areas in lungs, esophagus, intestines (FIG. 11) and heart (not shown) for these markers revealed no abnormalities in E18.5 or newborn Nurr1-mutant animals. In the walls of lung bronchi, neuronal innervation of smooth muscle was detected by pgp9.5 and acetylcholine analyses (FIG. 11A, G, M) and appeared normal in the mutant mice (FIGS. 11D, J, P). Also, in the esophagus and intestines, neuronal innervation was normal as detected by pgp9.5 immunoreactivity (FIGS. 11B, C, E and F), ACHE assay (FIGS. 11H, I, K, L) and VAChT immunoreactivity (FIGS. 11N, O, Q, R), in myenteric (Auerbach's) plexuses of external-muscle wall and the Meissner's plexuses of the submucosa. In the wild-type esophagus and intestines, VAChT immunoreactivity was also detected in the mucosal epithelium (FIGS. 11S and T). Notably, in the esophagus, VAChT was not detected to the same extent in the Nurr1 mutant pups (FIG. 11U), demonstrating an abnormality in this cell layer.

Deficient Ret mRNA expression in Nurr1 mutant mice in two defined brain nuclei identifies the first deregulated gene that is not directly related to dopaminergic neurotransmitter functions. Although Ret deficiency cannot explain the severe dopamine-secreting-cell developmental deficiencies observed in Nurr1^{-/-} dopaminergic neurons, the results link Nurr1 to novel functions associated with cell survival and other activities exerted by ligands utilizing Ret as their signaling-receptor subunit in cells where Nurr1 and Ret are coexpressed.

### Example 14

### LG268 Treatment of Nurr1^{+/+} and Nurr1^{-/-} Cultured Cells

Nurr1^{+/-} females were mated with Nurr1^{+/-} males to generate embryo litters of mixed genotypes (25%^{+/+}, 50%^{+/-} and 25%^{-/-}) as previously described (Zetterström et al, Science 1997). Pregnant females were sacrificed and embryos at embryonic stage E15.5 were dissected. Tails were removed for genotyping. Brains were fine-dissected and meninges removed. Both cortices from each embryo were removed and placed in 500 μl culture medium (N2). When all embryos in the litter were dissected, the cortices were triturated by mechanical dissociation using a fire-polished Pasteur pipette. Dissection of several litters revealed that plating of 50 μl cell suspension per well (24 well tissue culture plates pretreated with poly-D-lysine, rinsed and then preincubated with 400 μl N2) generated healthy-looking cultures. To avoid excessive cell death due to the extended dissection period when using gene targeted mice as compared to wild-type rats, 50 μl cell suspension was routinely plated. This generated cultures in the range of 200.000-470.000 cells per well. Six wells were plated per embryo; three were incubated in N2 only and three with N2+0.1 μM LG268. Cells were incubated for three days and thereafter fixed in 4% paraformaldehyde for 30 minutes and rinsed. For detection of neuronal nuclei, NeuN (diluted 1:300, Chemicon) was used in immunohistochemistry where visualization was with the ABC-kit from Vector. Cells were counted using brightfield microscopy (Eclipse E300M).

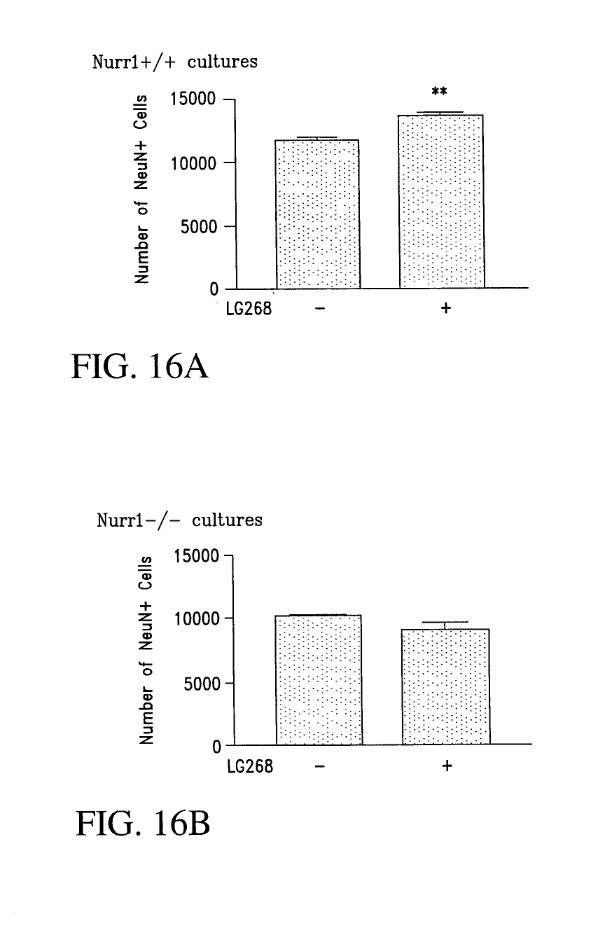

The involvement of Nurr1/RXR heterodimers in RXR-ligand promoted neuronal survival was tested by using primary cultures from wild-type (Nurr1^{+/+}) and Nurr1-knock-out mice (Nurr1^{-/-}). Cortical tissue was used since Nurr1 is not essential to the development of cortical neurons, as shown previously. Since the cortical cells from knock-out animals are deficient in Nurr1 expression, these cells could be detected using Nurr1 antibody. Also, however, since a relatively large proportion of cells in wild-type cortical cultures express Nurr1—about 30%—it is possible that the LG268 neurotrophic effect or enhanced survival of the cortical cells can be detected as an absolute increase in the total number of neurons. FIG. 16A shows that the total number of neurons, detected by NeuN immunoreactivity, are significantly increased after administration of LG268. In contrast, no increase was observed in cultures from knock-out mice. In conclusion, these data provide direct genetic evidence linking Nurr1 to the process of RXR ligand-induced neuronal survival.

Although the present invention has been described in detail with reference to the examples above, it is understood that various modifications can be made without departing from the spirit of the invention. All cited patents, patent applications and publications referred to in this application are herein incorporated by reference in their entirety.

We claim:

1. A method of increasing the survival of dopamine secreting cells, comprising the steps of administering a retinoid X receptor (RXR) ligand to dopamine secreting cells in an amount sufficient for the RXR ligand to bind RXR and increase the survival of the dopamine secreting cells.

2. The method of claim 1, wherein the RXR ligand selectively activates RXR.

3. The method of claim 1, wherein the dopamine secreting cells are cultured in vitro.

4. The method of claim 1, wherein the RXR ligand is a naturally-occurring ligand.

5. The method of claim 4, wherein the naturally-occurring ligand is isolated from ventral embryonic midbrain cells.

6. The method of claim 4, wherein the naturally-occurring ligand is 9-cis retinoic acid or docosahexaenoic acid.

7. The method of claim 1, wherein the RXR ligand is a non-naturally-occurring-RXR ligand.

8. The method of claim 7, wherein the non-naturally-occurring-RXR ligand is LG1069, LG100268, SR11203, SR11217, SR11234, SR11235, SR11236 or SR11237.

9. The method of claim 1, wherein the dopamine secreting cells are derived from the basal ganglia.

10. The method of claim 9, wherein the cells derived from the basal ganglia are derived from cells selected from the group consisting of: dopamine secreting cells from the striatum, globus pallidus, substantia innominate, ventral pallidum, nucleus basalis of Meynert, ventral tegmental area, the subthalmic nucleus, and the substantia nigra.

11. The method of claim 1, further comprising administering an RAR antagonist.

12. A method of increasing the survival of dopamine-secreting cells in a subject in need thereof, comprising the steps of administering a retinoid X receptor (RXR) ligand to the subject, in an amount sufficient for the RXR ligand to bind RXR and increase the survival of the dopamine-secreting cells.

13. The method of claim 12, wherein the subject has a neurodegenerative disease.

14. The method of claim 13, wherein the neurodegenerative disease is characterized by loss of dopamine-secreting cells.

15. The method of claim 14, wherein the neurodegenerative disease is Parkinson's disease.

16. The method of claim 12, wherein the RXR ligand selectively activates RXR.

17. The method of claim 12, wherein the RXR ligand is a naturally-occurring ligand.

18. The method of claim 17, wherein the naturally-occurring ligand is isolated from ventral embryonic midbrain cells.

19. The method of claim 17, wherein the naturally-occurring ligand is 9-cis retinoic acid or docosahexaenoic acid.

20. The method of claim 12, wherein the RXR ligand is a non-naturally-occurring-RXR ligand.

21. The method of claim 20, wherein the non-naturally-occurring-RXR ligand is LG1069, LG100268, SR11203, SR11217, SR11234, SR11235, SR11236 or SR11237.

22. The method of claim 12, wherein the dopamine-secreting cells are cells in the basal ganglia.

23. The method of claim 22, wherein the basal ganglia cells are located in the striatum, globus pallidus, substantia innominate, ventral pallidum, nucleus basalis of Meynert, ventral tegmental area, the subthalmic nucleus, or the substantia nigra.

24. The method of claim 12, further comprising administering an RAR antagonist.


SEQUENCE LISTING (1 sequence)

SEQ ID NO: 1 - protein, 11 aa, Homo sapiens
SKLLGKLPELR